(19) United States
(12) Patent Application Publication
(10) Pub. No.: US 2026/0216335 A1
(43) Pub. Date: Jul. 30, 2026

(54) COMPOSITIONS AND METHODS FOR TARGETING AND TREATING TUMOR

(71) Applicant: JIANGSU CODE BIOMEDICAL TECHNOLOGY CO., LTD., Nanjing (CN)

(72) Inventors: Shiyang Pan, Nanjing (CN); Jian Xu, Nanjing (CN); Lei Zhang, Nanjing (CN); Yue Pan, Ontario, CA (US)

(21) Appl. No.: 19/148,566

(22) PCT Filed: Jan. 17, 2024
(86) PCT No.: PCT/CN2024/072748

§ 371 Date: Jul. 16, 2025

(30) Foreign Application Priority Data
Jan. 17, 2023 (WO) PCT/CN2023/072658

(51) Int. Cl. A61K 41/00; A61K 47/68; A61K 47/69; A61K 49/00; B82Y 5/00

(52) U.S. Cl. CPC A61K 41/0052; A61K 47/6857; A61K 47/6923; A61K 47/6937; A61K 49/0034; A61K 49/0058; A61K 49/0093; B82Y 5/00

(57) ABSTRACT

Nanoparticles encapsulating core particles with attached photothermal agents, and therapeutic agents. The methods of imaging a tumor tissue and/or treating a subject suffering from tumor with the nanoparticles provided herein.

### TECHNICAL FIELD

This disclosure relates to compositions and methods for targeting and treating tumor. Specifically, nanoparticles encapsulating core particles (such as Fe₃O₄), photothermal agents, and therapeutic agents are provided. Also provided are methods of imaging a tumor tissue and/or treating subject having a tumor with the nanoparticles provided herein.

### BACKGROUND

Photothermal therapy (PTT) employs a near-infrared (NIR) laser and photo-absorbing agents to generate heat from light energy to “burn” cancer cells (see, e.g., Li, X., et al., Clinical development and potential of photothermal and photodynamic therapies for cancer. Nat Rev Clin Oncol, 2020. 17 (11): p. 657-674.). Photo-absorbing agents determine success or failure of PTT. Various NIR-absorbing materials, such as carbon nanotubes (see, e.g., Zhao, Y., et al., Temperature-Sensitive Lipid-Coated Carbon Nanotubes for Synergistic Photothermal Therapy and Gene Therapy. ACS Nano, 2021. 15 (4): p. 6517-6529), graphene oxide (see, e.g., Romero, M. P., et al., Graphene Oxide Theranostic Effect: Conjugation of Photothermal and Photodynamic Therapies Based on an in vivo Demonstration. Int J Nanomedicine, 2021. 16: p. 1601-1616) and gold nanostructures (see, e.g., Campu, A., et al., ICG-loaded gold nano-bipyramids with NIR activatable dual PTT-PDT therapeutic potential in melanoma cells. Colloids Surf B Biointerfaces, 2020. 194: p. 111213) have been detected in PTT application. However, nonbiodegradability and potential long-term toxicity restricted their clinical applications.

Therefore, improved compositions and methods for targeting and treating tumor using PTT are needed in the art.

### SUMMARY

This disclosure relates to nanoparticles encapsulating core particles (such as Fe₃O₄), photothermal agents, and/or therapeutic agents. The nanoparticles can further include an antigen-targeting molecule (e.g., coupled on the surface of the nanoparticles). Surface functionalization enhances the properties and characteristics of nanoparticles through surface modification, and enables them to play a major role in the field of medicine. In other words, nanoparticles functionalized with ligands exhibiting differential affinity towards proteins and cell surface molecules.

Accordingly, provided herein are compositions comprising: a photothermal agent, and one or more core particles, wherein the photothermal agent and the core particles are encapsulated in a nanoparticle made from a biodegradable material.

In some embodiments, the core particles are solid particles.

In some embodiments, the metal particles are Fe₃O₄ particles.

In some embodiments, the core particles are surface-modified for the attachment of the photothermal agent.

In some embodiments, the core particles are modified on the surface by oleic acid molecules.

In some embodiments, the photothermal agent is attached to the surface of the core particles through hydrophobic interactions with the oleic acid molecules.

In some embodiments, the core particles have a diameter of about 10 nm to about 40 nm.

In some embodiments, the composition comprises about 1 to about 10 core particles.

In some embodiments, the photothermal agent is selected from the group consisting indocyanine green (ICG), polydopamine, polyaniline (PANI), and polypyrrole (PPY).

In some embodiments, the photothermal agent is indocyanine green (ICG).

In some embodiments, the compositions described herein further comprise a therapeutic agent, wherein the therapeutic agent is encapsulated in the nanoparticle.

In some embodiments, the therapeutic agent is an anti-cancer therapeutic agent.

In some embodiments, the composition further comprises a tumor-targeting molecule attached to the surface of the nanoparticle.

In some embodiments, the tumor-targeting molecule is an antibody, or antigen-binding fragment thereof, that binds to a tumor-specific antigen.

In some embodiments, the diameter of the nanoparticle is about 70 nm to about 120 nm.

Also provided herein are methods of producing any one of the compositions described herein, wherein the method comprises: (a) contacting one or more core particles with the photothermal agent, thereby attaching the photothermal agent to the surface of the core particles; (b) preparing a double emulsion comprising the biodegradable material; and (c) contacting the product of step (a) with the double emulsion prepared in step (b), thereby producing the nanoparticles encapsulating the core particles; thereby producing the composition.

In some embodiments, the biodegradable material is poly (lactic-co-glycolic acid) (PLGA).

In some embodiments, the double emulsion further comprises polyvinyl alcohol (PVA).

In some embodiments, the method further comprises washing and/or freezing the nanoparticles.

Also provided herein are methods of imaging a tumor tissue in a subject in vivo, the method comprising: (a) administering an amount of any one of the compositions described herein to the subject; (b) exciting the photothermal agent of the composition; and (c) detecting the emitted fluorescent signals, thereby imaging the tumor tissue.

In some embodiments, the quality of the imaging is enhanced compared to an imaging without the administration of the composition.

Also provided herein are methods of diagnosing a tumor in a subject, the method comprising: (a) administering an amount of any one of the compositions described herein to the subject; (b) exciting the photothermal agent of the composition; and (c) detecting the presence or absence of emitted fluorescent signals, thereby diagnosing the tumor.

In some embodiments, the methods described herein further comprise recording the intensity of the emitted fluorescent signals.

Also provided herein are methods of treating a subject having tumor, the method comprising: (a) administering an amount of any one of the compositions described herein to the subject; (b) exciting the photothermal agent of the composition, thereby increasing the temperature of the tumor cells that has internalized the composition and damaging the tumor cells, thereby treating the subject having the tumor.

In some embodiments, the methods described herein further comprise treating the tumor with the therapeutic agent of the composition.

In some embodiments, wherein the therapeutic agent is a chemotherapy agent selected from the group consisting of paclitaxel, chlorambucil, cyclophosphamide, thiotepa, busulfan, purine antagonists, pyrimidine antagonists, folate antagonists, actinomycin D, doxorubicin, mitomycin, doxorubicin, mitoxantrone, and bleomycin. In some embodiments, the tumor is a solid tumor.

In some embodiments, the photothermal agent is excited using a near-infrared (NIR) light.

In some embodiments, the methods described herein further comprise targeting a specific tumor type by the tumor-targeting molecule of the composition.

In some embodiments, the tumor is lung cancer.

In some embodiments, the subject is a human subject.

In some embodiments, the administration is selected from the group consisting of intravenous injection, intraperitoneal injection, oral administration, oral transmucosal delivery, subcutaneous administration, intradermal administration, and transdermal administration.

In some embodiments, the photothermal agent is degraded after emitting heat.

In some embodiments, the heat emitted from the photothermal agent is transmitted to the core particles, thereby increasing the heating duration.

In some embodiments, the heating of the tumor cells induces the apoptosis of the tumor cells.

In some embodiments, the composition is internalized into the tumor cells through non-specific endocytosis.

In some embodiments, the composition is internalized into the tumor cells through specific recognition of tumor antigens.

In some embodiments, the heating of the tumor cells is repeated at least once. In some embodiments, the tumor cells are heated to about 40° C. to about 45° C.

Also provided herein are methods of evaluating a therapeutic effect in a subject undergoing treatment with one or more cancer therapy, the method comprising: (a) administering an amount of any one of the compositions described herein to the subject; (b) imaging a tumor area and determining the tumor size; (c) repeating the imaging and the determining of the tumor size at a later time point; and (d) evaluating the therapeutic effect based on the tumor size from the different time points; thereby evaluating the therapeutic effect in the subject.

In some embodiments, the methods described herein further comprise imaging and determining the tumor size before the subject is treated with the cancer therapy.

In some embodiments, the repeating of the imaging and determining of the tumor size is performed after about 1 week to about 1 year.

In some embodiments, the imaging is X-ray computed tomography (CT), magnetic resonance imaging (MRI), magnetic particle imaging (MPI), photoacoustic imaging (PA), ultrasound imaging (US), fluorescent imaging (FL), or a combination thereof.

Also provided here are uses of photothermal nanoparticles for the imaging a tumor tissue in a subject in vivo, comprising: (a) administering an amount of the composition described herein to the subject; (b) exciting the photothermal agent of the composition; and (c) detecting the emitted fluorescent signals, thereby imaging the tumor tissue.

Also provided herein are uses of photothermal nanoparticles for the diagnosis of a tumor in a subject, comprising: (a) administering an amount of the composition described herein to the subject; (b) exciting the photothermal agent of the composition; and (c) detecting the presence or absence of emitted fluorescent signals, thereby diagnosing the tumor.

Also provided herein are uses of photothermal nanoparticles for treating a subject having tumor, comprising: (a) administering an amount of the composition described herein to the subject; (b) exciting the photothermal agent of the composition, thereby increasing the temperature of the tumor cells that has internalized the composition and damaging the tumor cells, thereby treating the subject having the tumor.

Also provided herein are uses of photothermal nanoparticles for evaluating a therapeutic effect in a subject undergoing treatment with one or more cancer therapy, comprising: (a) administering an amount of the composition described herein to the subject; (b) imaging a tumor area and determining the tumor size; (c) repeating the imaging and the determining of the tumor size at a later time point; and (d) evaluating the therapeutic effect based on the tumor size from the different time points; thereby evaluating the therapeutic effect in the subject.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic illustration of the preparation and therapeutic functions of an example composition described herein (e.g., NJ001-ICG/OA Fe₃O₄@PLGA).

FIGS. 2A-2E show the characterization of nanoparticles. FIGS. 2A-2B show the SEM and TEM images of ICG/OA-Fe₃O₄@PLGA; FIG. 2C shows the hydrodynamic diameter; FIG. 2D shows the zeta potential of ICG/OA-Fe₃O₄@PLGA; and FIG. 2E shows UV-visible full wavelength scanning of free ICG, free OA-Fe₃O₄ and ICG/OA-Fe₃O₄@PLGA nanoparticles.

FIGS. 3A-3C show that ICG/OA-Fe₃O₄@PLGA has constant high photothermal conversion efficiency. FIG. 3A shows the temperature rising curve of various NPs solutions for 18 hours; and FIG. 3B shows the larger version for 0-4 hours. FIG. 3C shows the temperature maps captured at key time points (0 min, 1 min, 2 min, 3 min, 5 min, 10 min, 20 min, 30 min, 1 h, 2 h, 3 h, 5 h, 10 h, 15 h, 18 h) during the temperature monitoring process.

FIG. 4A show the temperature changes of ICG/OA-Fe₃O₄@NPs, Blank-NP, free ICG, ICG@PLGA and ICG/Fe₃O₄@PLGA at the same ICG concentration (0.012 mg/mL) under an 808 nm laser irradiation (1.0 W/cm²) for 51 cycles (3 min of irradiation followed by 7 min of cooling down for each cycle).

FIG. 4B shows the temperature maps captured at key time points (0 min to 113 min) during the temperature monitoring process of FIG. 4A.

FIGS. 5A-5B show the binding of tumor antigen-targeting nanoparticles with the SP70 antigen. NJ001-labeled or non-labeled PLGA nanoparticles were co-incubated with SPC-A1 culture supernatant containing SP70 for 30 min and then centrifuged. The remaining SP70 in supernatant were measured; (A) Blank-NP and NJ001-labeled Blank-NP (NJ001-NP) (B) ICG/OA-Fe₃O₄@PLGA and NJ001-ICG/OA-Fe₃O₄@PLGA.

FIG. 6 shows the detection of the binding ability of targeted nanoparticles to SP70 positive cells. The different lung cell lines with or without SP70 expression were cocultured with NJ001-ICG/OA-Fe₃O₄@PLGA or ICG/OA-Fe₃O₄@PLGA. After cocultured for 30 min, the cells were washed and continued to be cultured in their respective culture conditions. Surface and intracellular distributions of NJ001-ICG/OA-Fe₃O₄@PLGA in SPC-A1 cells were detected by confocal microscopy. NJ001-ICG/OA-Fe₃O₄@PLGA binding to SPC-A1 cells (high expression of SP70) was observed after coculturing for 30 min. As the time-lapse culturing continued, these NPs were gradually uptaken by SPC-A1.

FIG. 7A shows that the SPC-A1 cells (with high expression of SP70) had no binding and uptaking of ICG/OA-Fe₃O₄@PLGA.

FIG. 7B shows that the HBE cells (with no SP70 expression) had no binding and uptaking of NJ001-ICG/OA-Fe₃O₄@PLGA.

FIG. 8 shows the targeted heat-killing of tumor cells by NJ001-ICG/Fe₃O₄@PLGA. the nanoparticles were incubated with SPC-A1 cells for 30 min.

FIG. 9 shows the targeted heat-killing of tumor cells by NJ001-ICG/Fe₃O₄@PLGA. the nanoparticles were incubated with SPC-A1 cells for 30 min. After washed with culture medium, the culture was continued for 12 hours.

FIG. 10A shows the targeted heat-killing of tumor cells by NJ001-ICG/Fe₃O₄@PLGA. the nanoparticles were incubated with SPC-A1 cells for 30 min. After washed with culture medium, the culture was continued for 24 hours.

FIG. 10B shows the summary of results in FIGS. 8, 9, and 10A.

FIG. 11 shows the distribution of the nanoparticles in mice 1, 2, 3 and 10 days after the administration by inhaling.

FIG. 12A shows the temperature change of lung during irradiation. After the administration by inhaling, mice were irradiated for 2 consecutive days (first day and second day) at 808 nm, 2 W/cm² for 20 mins.

FIG. 12B shows the body temperatures of mice measured by an infrared temperature instrument at key time points. Legend: The lungs of mice were irradiated with 808 nm, 2 W/cm² near-infrared light, and the temperature maps at key time points (0, 2, 4, 6, 8, 10, 12, 14, 16, 18, 20 minutes) were captured.

FIGS. 13A-13B shows the tumor growth after nanoparticle administration and irradiation, compared with mice not administered the nanoparticles.

FIG. 14 shows the relative body weight after nanoparticle administration and irradiation, compared with mice not administered the nanoparticles.

FIGS. 15A-15B show the cancer nodule number in lung 21 days after nanoparticle administration and irradiation, compared with mice not administered the nanoparticles.

FIG. 16A shows serum content of SP70 (A) 0, 7, 14 and 21 days after nanoparticle administration, irradiation, and anti-tumor drug treatment.

FIG. 16B shows tissue expression of SP70 after 31 days of nanoparticle administration, irradiation, and anti-tumor drug treatment compared with mice not administered the nanoparticles.

FIG. 17 shows that NJ001-ICG/Fe₃O₄@PLGA injection did not cause toxic side effects in mice after administration, irradiation, and anti-tumor drug treatment, compared with mice not administered the nanoparticles.

FIG. 18 shows that NJ001-ICG/Fe₃O₄@PLGA injection did not affect liver and kidney functions in mice after administration, irradiation, and anti-tumor drug treatment, compared with mice not administered the nanoparticles.

### DETAILED DESCRIPTION

The disclosure provides a novel photothermal nanoparticle that is designed to achieve improved effects of imaging and killing of tumor tissues and cells. The photothermal agent encapsulated in the nanoparticles is efficiently attached to the surface of core particles, which can synergize with the photothermal agent and enhance and prolong the imaging of the tumor tissue. The nanoparticle also includes therapeutic agents that can be used for the treatment of cancer.

The compositions and methods described herein are effective in killing tumor cells and have low cytotoxicity. The nanoparticles are made from biodegradable materials and are especially suitable for in vivo applications.

The term “about,” particularly in reference to a given quantity, is meant to encompass deviations of plus or minus ten percent.

As used in this application, including the appended claims, the singular forms “a,” “an,” and “the” include plural references, unless the content clearly dictates otherwise, and are used interchangeably with “at least one” and “one or more.”

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements does not include only those elements but can include other elements not expressly listed or inherent to such process, method, product-by-process, or composition of matter.

By “consisting of” is meant including, and limited to, whatever follows the phrase “consisting of.” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the term “substantially” or “essentially” refers to a quantity, level, value, number, frequency, percentage, dimension, size, amount, weight or length that is about 90%, 91%, 92%, 93%, 94%, 95%, 96%, 97%, 98%, or 99% or higher compared to a reference quantity, level, value, number, frequency, percentage, dimension, size, amount, weight or length. In one embodiment, the terms “essentially the same” or “substantially the same” refer a range of quantity, level, value, number, frequency, percentage, dimension, size, amount, weight or length that is about the same as a reference quantity, level, value, number, frequency, percentage, dimension, size, amount, weight or length.

As used herein, the terms “substantially free of” and “essentially free of” are used interchangeably, and when used to describe a composition, such as a cell culture medium, refer to a composition that is free of a specified substance or its source thereof, such as, 95% free, 96% free, 97% free, 98% free, 99% free of the specified substance or its source thereof, or is undetectable as measured by conventional means. The term “free of” or “essentially free of” a certain ingredient or substance in a composition also means that no such ingredient or substance is (1) included in the composition at any concentration, or (2) included in the composition functionally inert, but at a low concentration. Similar meaning can be applied to the term “absence of,” where referring to the absence of a particular substance or its source thereof of a composition.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “patient,” as used herein preferably refers to a human, but also encompasses other mammals. It is noted that, as used herein, the terms “organism,” “individual,” “subject,” or “patient” are used as synonyms and interchangeably.

### Photothermal Nanoparticles

Provided herein are compositions comprising: a photothermal agent, and one or more core particles, wherein the photothermal agent and the core particles are encapsulated in a nanoparticle made from a biodegradable material.

Also provided herein are compositions comprising: (a) one or more core particles with a photothermal agent attached thereto; wherein the core particles are encapsulated in a nanoparticle made from a biodegradable material; and (b) one or more therapeutic agent.

The core particles described herein can be any suitable particles for the purpose of, e.g., carrying the photothermal agent, enhancing the imaging of the tumor tissue, and/or prolonging the imaging duration. In some embodiments, the core particles are solid particles. In some embodiments, the core particles are metallic oxide particles. In some embodiments, the metal particles are Fe₃O₄ particles, Al₂O₃, MgO, platinum particles, palladium particles, gold particles, manganese (Mn) particles, CuS particles, Ag₂S particles, FeS particles, MoS₂ particles, TiS₂ particles, WS₂ particles, FeSe₂ particles, MoOx particles (see, e.g., Zou et al., Polyethylene glycol-modified molybdenum oxide as NIR photothermal agent and its ablation ability for HeLa cells, February 2019 Colloid and Polymer Science 297 (1-10)), TiO₂ particles, or Ti₈O₁₅ particles. In some embodiments, the core particles are made from carbon nanotube (CNT). In some embodiments, the core particles are made from carbon quantum dots. In some embodiments, the core particles are made from graphene, or graphene oxide.

The core particles can be modified (e.g., surface-modified) to achieve better attachment with the photothermal agent. In some embodiments, the core particles are surface-modified for the attachment of the photothermal agent. In some embodiments, the core particles are modified on the surface by fatty acid (e.g., oleic acid) molecules. In some embodiments, the photothermal agent is attached to the surface of the core particles through hydrophobic interactions with the fatty acid (e.g., oleic acid) molecules. Any other suitable molecules can be used to modify the core particles as well. The attachment of the photothermal agent can also be achieved by any other suitable methods known in the art.

The size of the core particles is important for the efficacy and the safety of the use of the photothermal nanoparticles described herein. In some embodiments, the core particles have a diameter of about 10 nm, 15 nm, 20 nm, 25 nm, 30 nm, 35 nm, 40 nm, 45 nm, 50 nm, 55 nm, 60 nm, 65 nm, 70 nm, 75 nm, 80 nm, or more. In some embodiments, the core particles have a diameter of about 10 nm to about 40 nm. In some embodiments, the core particles have a diameter of about 10 nm to about 30 nm. In some embodiments, the core particles have a diameter of less than 40 nm. The size of the core particles can vary based on the material of the particles.

The core particles described herein, e.g., the Fe₃O₄ particles with a photothermal agent (e.g., indocyanine green) attached on the surface, are encapsulated in the photothermal nanoparticles described herein.

In some embodiments, the diameter of the photothermal nanoparticles described herein is about 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, or more. In some embodiments, the photothermal nanoparticles have a diameter of about 70 nm to about 120 nm.

In some embodiments, each photothermal nanoparticle encapsulates about 1 to about 50 core particles. In some embodiments, each photothermal nanoparticle encapsulates about 1 to about 40 core particles. In some embodiments, each photothermal nanoparticle encapsulates about 1 to about 30 core particles. In some embodiments, each photothermal nanoparticle encapsulates about 1 to about 20 core particles. In some embodiments, each photothermal nanoparticle encapsulates about 1 to about 10 core particles. In some embodiments, each photothermal nanoparticle encapsulates about 1 to about 5 core particles. In some embodiments, each photothermal nanoparticle encapsulates about 3 core particles. In some embodiments, each photothermal nanoparticle encapsulates about 5 to about 50 core particles. In some embodiments, each photothermal nanoparticle encapsulates about 5 to about 40 core particles. In some embodiments, each photothermal nanoparticle encapsulates about 5 to about 30 core particles. In some embodiments, each photothermal nanoparticle encapsulates about 5 to about 20 core particles. In some embodiments, each photothermal nanoparticle encapsulates about 5 to about 10 core particles. In some embodiments, each photothermal nanoparticle encapsulates about 10 to about 50 core particles. In some embodiments, each photothermal nanoparticle encapsulates about 10 to about 40 core particles. In some embodiments, each photothermal nanoparticle encapsulates about 10 to about 30 core particles. In some embodiments, each photothermal nanoparticle encapsulates about 10 to about 20 core particles.

The photothermal agent (also referred to as “photo-absorbing agent”) of the nanoparticles described herein is a critical component of the nanoparticles. Photothermal therapy (PTT) employs a near-infrared (NIR) laser and photo-absorbing agents to generate heat from light energy to “burn” cancer cells.

Any suitable photothermal agent known in the art can be used in the nanoparticles described herein. In some embodiments, the photothermal agent is indocyanine green (ICG), polydopamine, polyaniline (PANI), or polypyrrole (PPY).

In some embodiments, the photothermal agent is indocyanine green (ICG). Indocyanine green (ICG) is the only U.S. Food and Drug Administration (FDA) approved NIR clinical imaging agent. In addition, ICG shows excellent light-to-heat conversion efficiency which has potential value for hyperthermia. Nevertheless, liability to aggregate and degrade in aqueous solution, poor aqueous photostability, lack of target specificity, and rapid body clearance limit its application in PTT.

In some embodiments, the ICG encapsulated in the nanoparticles are degradable after repeated excitement. In some embodiments, the ICG encapsulated in the nanoparticles emits light and heat when excited, e.g., by NIR. In some embodiments, the heat emitted by the ICG encapsulated in the nanoparticles are transferred to the core particles it is attached to (e.g., the Fe₃O₄ particles), which in turn, prolongs the imaging duration and enhances the killing of the tumor cells.

The photothermal nanoparticles described herein further includes one or more therapeutic agent. In some embodiments, the therapeutic agent is encapsulated in the nanoparticle. In some embodiments, the therapeutic agent can also be attached on the surface of the nanoparticles.

The therapeutic agent in the nanoparticles are used for the killing of tumor cells and/or treating the tumor patient. Any suitable therapeutic agent can be used in the nanoparticles described herein.

In some embodiments, the therapeutic agent is an anti-cancer therapeutic agent. In some embodiments, the anti-cancer therapeutic agent is a chemotherapy drug.

In chemotherapy, cancer patients may be given one or several drugs from the available anti-cancer drugs. Since different chemical agents damage cancer cells in different ways and at different phases in the cell cycle, a combination of drugs is often employed to increase the cancerous cell-killing effectiveness. This is called combination chemotherapy.

Listed below are several major categories (classes) of chemotherapy agents based on their chemical structures and the way they act on cancer cells:

Alkylating agents were among the first anti-cancer drugs and are the most commonly used agents in chemotherapy today. Alkylating agents act directly on DNA, causing cross-linking of DNA strands, abnormal base pairing, or DNA strand breaks, thus preventing the cell from dividing. Alkylating agents are generally considered to be cell cycle phase nonspecific, meaning that they kill the cell in various and multiple phases of the cell cycle. Although alkylating agents may be used for most types of cancer, they are generally of greatest value in treating slow-growing cancers. Alkylating agents are not as effective on rapidly growing cells. Examples of alkylating agents include chlorambucil, cyclophosphamide, thiotepa, and busulfan.

Antimetabolites replace natural substances as building blocks in DNA molecules, thereby altering the function of enzymes required for cell metabolism and protein synthesis. In other words, they mimic nutrients that the cell needs to grow, tricking the cell into consuming them, so it eventually starves to death.

Antimetabolites are cell cycle specific. Antimetabolites are most effective during the S-phase of cell division because they primarily act upon cells undergoing synthesis of new DNA for formation of new cells. The toxicities associated with these drugs are seen in cells that are growing and dividing quickly. Examples of antimetabolites include purine antagonists, pyrimidine antagonists, and folate antagonists.

Plant alkaloids are antitumor agents derived from plants. These drugs act specifically by blocking the ability of a cancer cell to divide and become two cells. Although they act throughout the cell cycle, some are more effective during the S- and M-phases, making these drugs cell cycle specific. Examples of plant alkaloids used in chemotherapy are actinomycin D, doxorubicin, and mitomycin.

Antitumor antibiotics are cell cycle nonspecific. They act by binding with DNA and preventing RNA (ribonucleic acid) synthesis, a key step in the creation of proteins, which are necessary for cell survival. They are not the same as antibiotics used to treat bacterial infections. Rather, these drugs cause the strands of genetic material that make up DNA to uncoil, thereby preventing the cell from reproducing. Doxorubicin, mitoxantrone, and bleomycin are some examples of antitumor antibiotics.

The nanoparticles described herein can be tumor-specific or non-tumor-specific. To be specific to a certain tumor type, or a subgroup of tumor cells, the nanoparticles can include a tumor antigen-specific molecule for the recognition of the specific type of tumor or tumor cells.

In some embodiments, the nanoparticles described herein include a tumor-targeting molecule attached to the surface of each nanoparticle. The tumor-targeting molecule can be any suitable kind of tumor cell-recognizing molecule known in the art.

In some embodiments, the tumor-targeting molecule is an antibody, or antigen-binding fragment thereof, that binds to a tumor-specific antigen.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope in an antigen. Non-limiting examples of antibodies include: monoclonal antibodies, polyclonal antibodies, multi-specific antibodies (e.g., bi-specific antibodies), single-chain antibodies, single variable domain (V_{H}H) antibodies, chimeric antibodies, human antibodies, and humanized antibodies. In some embodiments, an antibody can contain an Fc region of a human antibody. The term antibody also includes derivatives, e.g., multi-specific antibodies, bi-specific antibodies, single-chain antibodies, diabodies, and linear antibodies formed from these antibodies or antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain, a variable domain of light chain or a V_{H}H). Non-limiting examples of antibody fragments include, e.g., Fab, Fab′, F(ab′) 2, and Fv fragments, scFv, and V_{H}H.

As used herein, when referring to an antibody or an antigen-binding fragment, the phrases “specifically binding” and “specifically binds” mean that the antibody or an antigen-binding fragment interacts with its target molecule preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general.

In some embodiments, the antibody, or antigen-binding fragment thereof attached to the surface of the nanoparticles is an Sp70-specific antibody, or antigen-binding fragment thereof. In some embodiments, the antibody, or antigen-binding fragment thereof is NJ001 or a fragment thereof (see, e.g., Pan et al., PLOS One, 2012; 7 (3): e33009. doi: 10.1371/journal. pone. 0033009. Epub 2012 Mar. 30).

The nanoparticles of the composition provided herein (e.g., photothermal nanoparticles) can be made from any suitable polymer. In some embodiments, the photothermal nanoparticles are made from a biodegradable material. In some embodiments, the photothermal nanoparticles are made from poly (lactic-co-glycolic acid) (PLGA). In some embodiments, the photothermal nanoparticles are made from poly (lactic acid) (PLA). In some embodiments, the photothermal nanoparticles are made from liposome. In some embodiments, the photothermal nanoparticles are made from chitosan. In some embodiments, the photothermal nanoparticles are made from a hydrogel.

In some embodiments, provided herein is a composition including a photothermal nanoparticle encapsulating one or more oleic acid-modified Fe₃O₄ core particles with ICG attached thereto, and a therapeutic agent, wherein the photothermal nanoparticles have antibody NJ001 attached to the surface.

### Methods of Producing Nanoparticles

In one aspect, provided herein is a method of producing the compositions (e.g., photothermal nanoparticles) described herein, wherein the method comprises: (a) contacting one or more core particles with the photothermal agent, thereby attaching the photothermal agent to the surface of the core particles; (b) preparing a double emulsion comprising the biodegradable material; and (c) contacting the product of step (a) with the double emulsion prepared in step (b), thereby producing the nanoparticles encapsulating the core particles; thereby producing the composition.

In some embodiments, the double emulsion further comprises polyvinyl alcohol (PVA). In some embodiments, the method further includes washing and/or freezing the nanoparticles. Any other suitable methods of producing nanoparticles can be used to produce the nanoparticles described herein.

Any suitable methods can be used to produce the nanoparticles described herein. Methods of producing nanoparticles are known in the art.

In some embodiments, the nanoparticles are produced using a phase separation method (see, e.g., Husmann M, et al., Polymer erosion in PLGA microparticles produced by phase separation method. Int J Pharm. 2002 Aug. 21; 242 (1-2): 277-80)

In some embodiments, the nanoparticles are produced using a nanoparticles can salting-out method (see, e.g., Wang et al., Manufacturing Techniques and Surface Engineering of Polymer Based Nanoparticles for Targeted Drug Delivery to Cancer February 2016 Nanomaterials 6 (2): 26).

In some embodiments, the nanoparticles are produced using a spray drying method (see, e.g., Chopde et al., Nanoparticle formation by nanospray drying & its application in nanoencapsulation of food bioactive ingredients, Journal of Agriculture and Food Research, Volume 2, 2020, 100085, ISSN 2666-1543,).

In some embodiments, the nanoparticles are produced using a membrane emulsification method (see, e.g., Joseph S, et al., Preparation of nanoemulsions and solid lipid nanoparticles by premix membrane emulsification. J Pharm Sci. 2012 July; 101 (7): 2479-89).

In some embodiments, the nanoparticles are produced using a nanoprecipitation method (see, e.g., Yadav K S et al., Modified nanoprecipitation method for preparation of cytarabine-loaded PLGA nanoparticles. AAPS PharmSciTech. 2010 September; 11 (3): 1456-65. doi: 10.1208/s12249-010-9519-4. Epub 2010 Sep. 15.).

In some embodiments, the nanoparticles are produced using a supercritical fluid method (see e.g., Byrappa K et al., Nanoparticles synthesis using supercritical fluid technology-towards biomedical applications. Adv Drug Deliv Rev. 2008 Feb. 14; 60 (3): 299-327. doi: 10.1016/j, addr. 2007.09.001. Epub 2007 Oct. 12.).

In some embodiments, the photothermal agent (e.g., indocyanine green (ICG)) is attached to the core particles (e.g., oleic acid modified ferric oxide, Fe₃O₄) before the production of the nanoparticles.

In some embodiments, the biodegradable materials, e.g., PLGA polymer, is dissolved in a solvent, e.g. dichloromethane. After completely dissolved, it is added to the photothermal agent (e.g., ICG solution). In some embodiments, a colostrum is produced. In some embodiments, a second polymer (e.g., 2% polyvinyl alcohol (PVA) solution) is added to the colostrum to make the double emulsion. In some embodiments, the microspheres are solidified. In some embodiments, the nanoparticles are washed, centrifuged, freeze-dried and/or stored (e.g., at 4° C.) for later use.

### Imaging of Tumor Tissues

Also provided herein are methods of imaging a tumor tissue in a subject in vivo, the method comprising: (a) administering an amount of the photothermal nanoparticles to the subject; (b) exciting the photothermal agent of the composition; and (c) detecting the emitted fluorescent signals, thereby imaging the tumor tissue.

As used herein, the terms “subject” and “patient” are used interchangeably throughout the specification and describe an animal, human or non-human, to whom imaging and/or treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

In some embodiments, the subject is a human subject. In some embodiments, the subject is a tumor patient.

Fluorescence imaging is a rapidly evolving modality used in the intraoperative setting to delineate the vasculature, lymphatic drainage, or demarcate between tumor and normal tissue. Indocyanine green (ICG) is a water-soluble dye with a peak spectral absorption and emission at 800 to 810 nm in the blood or plasma. ICG has been used for angiography, to measure cardiac output or cerebral blood flow, evaluate hepatic function, and assess regional blood flow assessment. After intravenous administration, the dye is bound by plasma proteins, remaining intravascular, thereby allowing blood vessels to be visualized when the tissue is illuminated and observed at appropriate wavelengths of light.

By incorporating photothermal agents such as ICG into the nanoparticles described herein, tumor tissues can be specifically targeted, e.g., by the tumor antigen-targeting molecules on the surface of the nanoparticles, and imaged. In some embodiments, the photothermal agent is excited by fluorescent light. In some embodiments, the fluorescent light is laser-induced fluorescence. In some embodiments, the photothermal agent is excited using a near infrared (NIR) light. In some embodiments, the imaging is X-ray computed tomography (CT).

In some embodiments, the method described herein includes imaging the tumor tissue using any method known in the art. In some embodiments, the imaging is magnetic resonance imaging (MRI). In some embodiments, the imaging is magnetic particle imaging (MPI). In some embodiments, the imaging is photoacoustic imaging (PA). In some embodiments, the imaging is ultrasound imaging (US). In some embodiments, the imaging is fluorescent imaging (FL). In some embodiments, the quality of the imaging is enhanced compared to an imaging without the administration of the composition.

The imaging can be achieved using any nanoparticle described herein. One or more dyes, such as IR-780, IR-808, IR825, cypate, and/or porphyrin can be included in any of the nanoparticles described herein for the imaging using corresponding irradiation method.

In some embodiments, the tumor tissue is specifically targeted and imaged. In some embodiments, the nanoparticles do not specifically target a tumor type and primarily enters a specific organ, thereby enabling the imaging of the specific organ/tissue.

In some embodiments, the fluorescence is applied for about 10 min to about 30 min. In some embodiments, the fluorescence is applied for about 10 min.

### Methods of Treatment

Also provided herein is a method of treating a subject having tumor, the method comprising administering an amount of the composition comprising the photothermal nanoparticles described herein to the subject.

Also provided herein is a method of treating a subject having tumor, the method comprising: (a) administering an amount of the composition comprising the photothermal nanoparticle described herein to the subject; and (b) exciting the photothermal agent of the composition, thereby increasing the temperature of the tumor cells that has internalized the composition and damaging the tumor cells, thereby treating the subject having the tumor.

In some embodiments, the method further includes administering an anti-cancer therapy to the subject.

In some embodiments, the excitement of the photothermal agent in the nanoparticles increases the temperature of the tumor cells that has internalized the nanoparticles and thereby damaging or killing the tumor cells. In some embodiments, the heating of the tumor cells is repeated at least once.

In some embodiments, the photothermal agent is degraded after repeated excitement. The degradable photothermal agent in the nanoparticle also improves the safety and biocompatibility of the use of the nanoparticles for imaging and treatment of tumors.

In some embodiments, the heat emitted from the photothermal agent is transmitted to the core particles, thereby increasing the heating duration. In some embodiments, the tumor cells are heated to about 40° C. to about 55° C. In some embodiments, the tumor cells are heated to about 40° C. to about 50° C. In some embodiments, the tumor cells are heated to about 40° C. to about 45° C. In some embodiments, the tumor cells are heated to about 45° C. to about 55° C. In some embodiments, the tumor cells are heated to about 45° C. to about 50° C. In some embodiments, the tumor cells are heated to about 50° C. to about 55° C. In some embodiments, the tumor cells are heated to about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., about 55° cor more. In some embodiments, the tumor cells are heated to about 51.9° C.

In some embodiments, the tumor cells are heated to the temperature described herein within about 10 seconds, 20 seconds, 30 seconds, 40 seconds, 50 seconds, 1 min, 2 min, 3 min, 4 min, 5 min, 6 min, 7 min, 8 min, 9 min, 10 min, 11 min, 12 min, 13 min, 14 min, 15 min, 16 min, 17 min, 18 min, 19 min, 20 min, 25 min, 30 min, 35 min, 40 min or more. In some embodiments, the tumor cells are heated to about 51.9° C. within about 3 min.

In some embodiments, the temperature of the heated tumor cells is maintained at an increased temperature (e.g., about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C.) for a certain duration for the killing of the tumor cells. In some embodiments, the temperature of the heated tumor cells is maintained above about 40° C., about 41° C., about 42° C., about 43° C., about 44° C., about 45° C., about 46° C., about 47° C., about 48° C., about 49° C., about 50° C., about 51° C., about 52° C., about 53° C., about 54° C., or about 55° C.

In some embodiments, the temperature is maintained for about 0.5 hour, 1 hour, 1.5 hour, 2 hours, 2.5 hours, 3 hours, 3.5 hours, 4 hours, 4.5 hours, 5 hours, 5.5 hours, 6 hours, 6.5 hours, 7 hours, 7.5 hours, 8 hours, 8.5 hours, 9 hours, 9.5 hours, 10 hours, 11 hours, 12 hours or more. In some embodiments, the temperature is maintained for about 2.5 hours. Any other duration of heating and maintaining of the heated temperature can be used in the method described herein. In some embodiments, the temperature of the heated tumor cells is maintained at above about 45° C. for about 2.5 hours.

In some embodiments, the method of treatment described herein induces apoptosis of tumor cells.

In some embodiments, the compositions are administered to the subject without exciting (e.g., irradiation) of the photothermal agent and the percentage of apoptosis induced by the method is about 5%, 10%, 15%, 20%, 25%, 30%, 35%, 40%, 45%, 50%, 55%, 60%, 65%, 70%, 75%, 80%, 85%, 90% or more of the tumor cells. In some embodiments, the compositions are administered to the subject without exciting (e.g., irradiation) of the photothermal agent and the percentage of apoptosis induced by the method is about 35% (e.g., 35.0±3.5%).

In some embodiments, the compositions are administered to the subject and the photothermal agent is excited (e.g., irradiated using NIR). The percentage of apoptosis induced by the method is about 5%, 10%, 15%, 20%, 25%, 30%, 35%, 40%, 45%, 50%, 55%, 60%, 65%, 70%, 75%, 80%, 85%, 90% or more of the tumor cells.

The percentage of apoptosis induced by the administration of the composition and irradiation of the photothermal agent is about 83% (83.5±2.8%).

In some embodiments, the percentage of apoptosis of the tumor cells induced by administering the composition and exciting the photothermal agent is higher than the percentage of apoptosis of the tumor cells induced by administering the composition without exciting of the photothermal agent (e.g., by irradiation using NIR).

In some embodiments, the apoptosis is measured about 1 hour to about 36 hours after the administration. In some embodiments, the apoptosis is measured within about 24 hours after the administration.

In some embodiments, the heating of the tumor cells induces the apoptosis of the tumor cells.

In some embodiments, the nanoparticles are internalized into the tumor cells through non-specific endocytosis. In some embodiments, the nanoparticles are internalized into the tumor cells through specific recognition of tumor antigens.

Any suitable anti-cancer therapeutic agent can be used in the method described herein. In some embodiments, the therapeutic agent is a chemotherapy agent selected from the group consisting of chlorambucil, cyclophosphamide, thiotepa, busulfan, 5-fluorouracil, cis-platinum, paraplatin, docetaxel, pemetrexed, PTX (paclitaxel), purine antagonists, pyrimidine antagonists, folate antagonists, actinomycin D, doxorubicin, mitomycin, doxorubicin, mitoxantrone, and bleomycin.

In some embodiments, the methods further includes using additional therapies in combination with the administration of the anti-cancer therapeutic agent. The additional therapies can include chemotherapy, immunotherapy, targeted cancer therapy, a monoclonal antibody, an anti-HER2 antibody, trastuzumab, an anti-VEGF antibody, bevacizumab, and/or platinum/taxane therapy. In some embodiments, the additional treatment comprises at least one of afatinib, afatinib+cetuximab, alectinib, aspirin, atezolizumab, bicalutamide, cabozantinib, capecitabine, carboplatin, ceritinib, cetuximab, cisplatin, crizotinib, dabrafenib, dacarbazine, doxorubicin, enzalutamide, epirubicin, erlotinib, everolimus, exemestane+everolimus, fluorouracil, fulvestrant, gefitinib, gemcitabine, hormone therapies, irinotecan, lapatinib, liposomal-doxorubicin, matinib, mitomycin-c, nab-paclitaxel, nivolumab, olaparib, osimertinib, oxaliplatin, palbociclib combination therapy, paclitaxel, palbociclib, panitumumab, pembrolizumab, pemetrexed, pertuzumab, sunitinib, T-DM1, temozolomide docetaxel, temsirolimus, topotecan, trametinib, trastuzumab, vandetanib, and vemurafenib. The hormone therapy can be one or more of tamoxifen, toremifene, fulvestrant, letrozole, anastrozole, exemestane, megestrol acetate, leuprolide, goserelin, bicalutamide, flutamide, abiraterone, enzalutamide, triptorelin, abarelix, and degarelix.

In some embodiments, the anti-tumor drug is a small molecule drug. In some embodiments, the anti-tumor drug is JQ1 (see, e.g., Jiang G, Deng W, Liu Y and Wang C: General mechanism of JQ1 in inhibiting various types of cancer. Mol Med Rep 21:1021-1034, 2020).

In some embodiments, the tumor is a solid tumor. Solid tumors include without limitation lung cancer, non-small cell lung cancer small cell lung cancer (including small cell carcinoma (oat cell cancer), mixed small cell/large cell carcinoma, and combined small cell carcinoma), colon cancer, breast cancer, prostate cancer, liver cancer, pancreatic cancer, brain cancer, kidney cancer, ovarian cancer, stomach cancer, melanoma, bone cancer, gastric cancer, glioma, glioblastoma, hepatocellular carcinoma, papillary renal carcinoma, head and neck squamous cell carcinoma, thyroid carcinoma, leukemia, lymphoma, myeloma, or other solid tumors.

In some embodiments, the methods of treating a tumor described herein is combined with one or more imaging methods described herein or known in the art. In some embodiments, the methods of treating a tumor described herein is combined with one or more additional cancer therapies known in the art.

Also provided herein is a method of diagnosing a tumor in a subject, the method comprising: (a) administering an amount of the photothermal nanoparticles to the subject; and (b) detecting the presence or absence of a signal from the nanoparticles, thereby diagnosing the tumor.

In some embodiments, the method further includes exciting the photothermal agent in the nanoparticles (e.g., using NIR). Any other detection (e.g., imaging) methods can be used in the methods described herein. In some embodiments, the signal is a fluorescent signal.

Also provided herein is a method of diagnosing a tumor in a subject, the method comprising: (a) administering an amount of the photothermal nanoparticles to the subject; (b) exciting the photothermal agent of the composition; and (c) detecting the presence or absence of emitted fluorescent signals, thereby diagnosing the tumor.

In some embodiment, the method further includes recording the intensity of the emitted fluorescent signals. In some embodiments, the presence or absence of the tumor is indicated by the fluorescence emitted by the photothermal agent.

In the methods described herein, the photothermal nanoparticles can be administered via any suitable route. In some embodiments, the administration is intravenous injection, intraperitoneal injection, intramuscular injection, intrathecal administration, oral administration, oral transmucosal delivery, subcutaneous administration, intradermal administration, or transdermal administration. In some embodiments, the photothermal nanoparticles are administered by inhalation.

Depending on the type of cancer and the kind of drug used, the additional therapeutic anti-cancer drugs may be administered differently. They can be administered orally (oral chemotherapy), or injected into a muscle (intramuscular injection), injected under the skin (subcutaneous injection), or into a vein (intravenous chemotherapy). In special cases, chemotherapy drugs may be injected into the fluid around the spine (intrathecal chemotherapy). Two or more methods of administration may be used at the same time under certain circumstances. No matter what method is used, chemotherapy drugs are absorbed into the blood and carried around the body.

Of all the methods of chemotherapy drug administration mentioned above, intravenous injection is most commonly used. It is the most efficient way to get the medication into the bloodstream. Oral chemotherapy is more convenient and does not require any specialized equipment.

In some embodiments, the methods described herein can be performed in combination with one or more additional cancer therapies. Any suitable cancer therapies can be used in combination with the methods described herein.

For targeted cancer therapy, specific tumor markers are targeted, e.g., through the tumor antigen-specific molecules on the surface of the nanoparticles. In some embodiments, the tumor antigen-specific molecule is an antibody, or a fragment thereof that specifically bind to a tumor antigen. For example, the tumor antigen-specific molecule can be an anti-SP70 antibody.

A tumor marker is anything present in or produced by cancer cells or other cells of the body in response to cancer or certain benign (noncancerous) conditions that provides information about a cancer, such as how aggressive it is, whether it can be treated with a targeted therapy, or whether it is responding to treatment.

Tumor markers that are in common use are known in the art.

Tumor antigens (or “cancer antigens” used interchangeably) are antigens that are associated with one or more types of tumor. A tumor antigen is an antigenic substance produced in tumor cells, i.e., it triggers an immune response in the host. Tumor antigens are useful tumor markers in identifying tumor cells with diagnostic tests and are potential candidates for use in cancer therapy. Numerous tumor-associated antigens are known in the art. Exemplary tumor-associated antigens include, but are not limited to, 5 alpha reductase, alpha-fetoprotein, AM-1, APC, April, BAGE, beta-catenin, Bc112, bcr-abl, CA-125, CASP-8/FLICE, Cathepsins, CD19, CD20, CD21, CD23, CD22, CD33 CD35, CD44, CD45, CD46, CD5, CD52, CD55, CD59, CDC27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pme117, gp-100-in4, gp15, gp75/TRP-1, hCG, heparanse, Her2/neu, HMTV, HSP70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, uPA, PRAME, probasin, progenipoientin, PSA, PSM, RAGE-1, Rb, RCAS1, SART-1, SSX-family, STAT3, STn, TAG-72, TGF-alpha, TGF-beta, Thymosin-beta-15, TNF-alpha, TYRP-, TYRP-2, tyrosinase, VEGF, ZAG, p16INK4, and glutathione-S-transferase.

The nanoparticles described herein can also be used in companion diagnostics and/or treatment. The level of the tumor markers (e.g., SP70) can be detected in both the tumor tissues and in bodily fluids (e.g., peripheral blood). In some embodiments, the targeted molecule (e.g., SP70) participates in the immune responses involved in cancer. The nanoparticles described herein including the anti-tumor agent can then be used to target the tumor marker (e.g., SP70) and reduce the level of the targeted molecule in tumor tissue or a bodily fluid. The nanoparticles thus serves as a therapeutic agent for the alleviation and treatment of cancer.

In some embodiments, the nanoparticles described herein are used for the diagnosis and treatment of a liquid tumor. In some embodiments, the liquid tumor is leukemia. In some embodiments, one of the tumor marker for the liquid tumor is SP70. Leukemia is a hematological malignancy that originates from hematopoietic stem cells in the bone marrow. Leukemia usually involves the white blood cells. Leukemia starts in blood-forming tissues such as the bone marrow. In patients with leukemia, the bone marrow produces an excessive amount of abnormal white blood cells. Acute myeloid leukemia (AML) can spread to spinal fluid and other parts of the central nervous system (CNS) (Yamaguchi T et al., Remodeling of Bone Marrow Niches and Roles of Exosomes in Leukemia. Int J Mol Sci. 2021 Feb. 13; 22 (4): 1881).

Therefore, in some embodiments, the nanoparticles described herein can be used to diagnose a liquid tumor (e.g., leukemia) by targeting one or more liquid tumor specific antigen (e.g., SP70) and via imaging. In some embodiments, the nanoparticles described herein can be used to treat a liquid tumor (e.g., leukemia) by targeting one or more liquid tumor specific antigen (e.g., SP70) and including one or more anti-cancer agents in the nanoparticle. The administration of the nanoparticles can be local administration or systemic administration. In some embodiments, the nanoparticles target the leukemia cancer cells in bone marrow. In some embodiments, the nanoparticles can target the leukemia cancer cells in spinal fluid.

The photothermal nanoparticles described herein can further serve as a light source for operations for cancer treatment. In some embodiments, the fluorescence signal emitted from the nanoparticles in the tumor area guides a tumor resection surgery. Any suitable operation methods for tumor removal can be used in the methods described herein. In some embodiments, the tumor resection surgery is performed using a fiberscope. Because of the advantages of enhanced tumor imaging provided by the nanoparticles described herein, the tumor resection surgeries can be performed in a minimally invasive way.

### Methods of Evaluating Therapeutic Effects

Also provided herein are methods of evaluating a therapeutic effect of a cancer treatment. The nanoparticles provided herein are administered and distributed in tumor tissues (through targeted or non-specific distribution) and can be imaged for a duration of time to monitor the therapeutic effect of a treatment.

Accordingly, in one aspect, provided herein is a method of evaluating a therapeutic effect in a subject undergoing treatment with one or more cancer therapy, the method comprising: (a) administering an amount of the composition as described herein to the subject; (b) imaging a tumor area and determining the tumor size; (c) repeating the imaging and the determining of the tumor size at a later time point; and (d) evaluating the therapeutic effect based on the tumor size from the different time points; thereby evaluating the therapeutic effect in the subject.

In some embodiments, the method further includes imaging and determining the tumor size before the subject is treated with the cancer therapy.

In some embodiments, the imaging includes exciting the photothermal agent in the photothermal nanoparticles (e.g., using NIR). Other suitable imaging techniques can also be used for the imaging.

In some embodiments, the therapeutic effect is evaluated for a duration of about 1, 2, 3, or 4 weeks. In some embodiments, the therapeutic effect is evaluated for a duration of about 1, 2, 3, 4, 5, 6, 7, 8, 10, 11, or 12 months.

In some embodiments, the therapeutic effect is evaluated by imaging a tumor sample (e.g., a tissue sample from a tumor tissue). The frequency of the evaluation can be any proper duration and the evaluation can be performed based on the patient's specific conditions. For example, the therapeutic effect can be evaluated about every week, every two weeks, every three weeks, every four weeks, every five weeks, every six weeks, every seven weeks, every eight weeks, every nine weeks, every ten weeks, every eleven weeks, every twelve weeks or more. The therapeutic effect can be evaluated about every month, every two months, every three months, every four months, every five months, every six months, every seven months, every eight months, every nine months, every ten months, every eleven months, every year, every two years, every three years or more.

In some embodiments, the therapeutic effect is evaluated by measuring the level of one or more tumor antigen(s) in a biological sample from the subject. In some embodiments, the biological sample is a bodily fluid, e.g., blood or plasma. In some embodiments, the therapeutic effect is evaluated by measuring the level of one or more circulating tumor antigen(s) in a bodily fluid (e.g., blood or plasma) from the subject. For example, the level of circulating SP70 can be measured before and after the treatment for the evaluation of a therapeutic effect of a cancer.

The evaluation of the therapeutic effect can be performed using any suitable method and criteria. In some embodiments, the therapeutic effect is evaluated by the size of the tumor tissue area. In some embodiments, the tumor size is reduced (e.g., after performing the method of treating a cancer described herein) by about 1%, 5%, 10%, 15%, 20%, 25%, 30%, 35%, 40%, 45, 50%, 55%, 60%, 65%, 70%, 75%, 80%, 85%, 90%, 95%, 96%, 97%, 98%, 99%, or more, compared to the tumor size before the cancer treatment.

The evaluation of the size of the tumor tissue area can be performed using any imaging method described herein or known in the art. In some embodiments, the tumor size is measured using enhanced CT.

### EXAMPLES

The invention is further described in the following examples, which do not limit the scope of the invention described in the claims.

With an eye forward targeted delivery and clinical applications. We developed a NJ001 labeled biodegradable nanocarrier system encapsulated ICG and oleic acid modified Fe₃O₄. NJ001 is an monoclonal antibody (mAb) specific to SP70, which is a novel tumor marker of non-small-cell lung cancer (NSCLC), especially lung adenocarcinoma. NJ001-labeled probes have a strong tumor targeting effect. Fe₃O₄ is also approved by FDA for use in biomedical field and has been widely employed in targeted drug carriers (see, e.g., Nikolaou, M., et al., Superparamagnetic electrospun microrods for magnetically-guided pulmonary drug delivery with magnetic heating. Mater Sci Eng C Mater Biol Appl, 2021. 126: p. 112117), nuclear magnetic resonance (see, e.g., Kang, N., et al., Magnetic targeting core/shell Fe₃O₄/Au nanoparticles for magnetic resonance/photoacoustic dual-modal imaging. Mater Sci Eng C Mater Biol Appl, 2019. 98: p. 545-549) and tissue engineering (see, e.g., Lai, W. Y., et al., In Vivo Investigation into Effectiveness of Fe(₃)O(₄)/PLLA Nanofibers for Bone Tissue Engineering Applications. Polymers (Basel), 2018.10 (7)). Especially, oleic acid modified Fe₃O₄ (OA-Fe₃O₄) showed a strong ICG carrying capability by direct adherence of ICG.

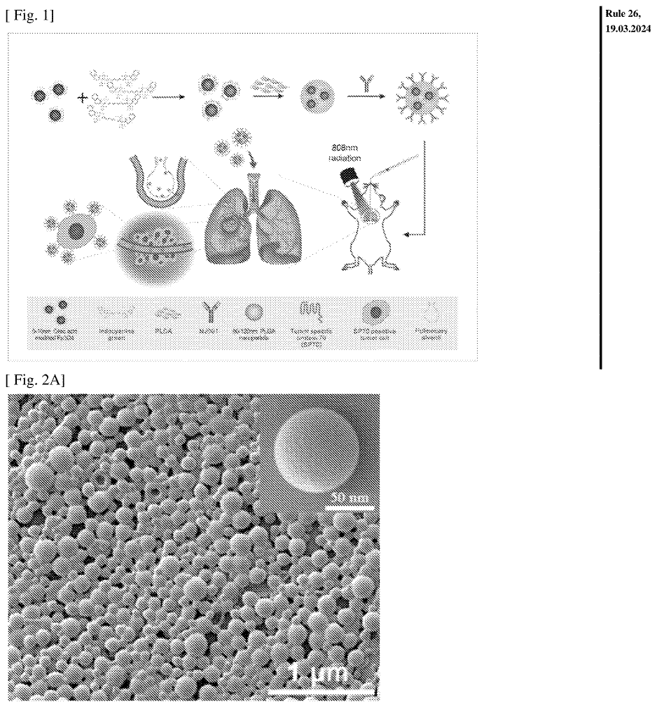

In this study, ICG loaded on the oleic acid modified Fe₃O₄ nanoparticles (denoted as OA-Fe₃O₄/ICG) via hydrophobic interactions between the oleic acid branch and ICG. Subsequently, OA-Fe₃O₄/ICG was encapsulated by PLGA (denoted as OA-Fe₃O₄/ICG@NP) to fabricated a nanoparticle with sufficient carboxyl groups on their surfaces. After phenotype and photothermal conversion efficiency were analyzed, OA-Fe₃O₄/ICG@NP coupled with NJ001 to construct a novel target nanoparticle. The novel tumor marker SP70 targeted nanoparticle (NJ001-OA-Fe 304/ICG@NP) was comprehensively characterized and intravenously administered into lung adenocarcinoma mice model. Its targeted ability and therapeutic effect were systematically evaluated by bioluminescence imaging, fluorescence imaging and micro-computed tomography (micro-CT). FIG. 1 is a schematic illustration of the production of the nanoparticles and the use of the nanoparticles for the treatment of cancer in a mouse model.

### Example 1. Material and Methods

### Material, Mice and Cell Lines

Indocyanine green (ICG) (purchased from Aladdin). Oleic acid modified ferric oxide (purchased from Nanjing Shennuoqing Biotechnology Co. Ltd.). PLGA (purchased from Sigma Aldrich). Thirty-four Four-week-old male BALB/c nude mice (purchased from Shanghai SLAC Laboratory Animal Co. Ltd. Shanghai, China). SPC-A1-luc cells stably expressing luciferase (purchased from Shanghai Baidai Biotechnology Co. Ltd.). RPMI 1640, Fetal bovine serum (purchased from Gibco), Luciferase substrate (purchased from Beyotime).

Fabrication of PLGA-NPs with Encapsulated ICG and OA-Fe₃O₄

PLGA polymer (50 mg) was dissolved in 1.25 mL dichloromethane. After completely dissolved, it was added into 200 μL 10 mg/mL ICG solution. The ultrasonic crusher worked for 60 seconds at 100 W power to produce colostrum. Adding 30 ml of 2% polyvinyl alcohol (PVA) solution, the ultrasonic crusher worked at 100 W power for 100 s to make the double emulsion. More PVA was added to the emulsion solution and stirred overnight to volatilize methylene chloride and solidify the microspheres. Finally, the nanoparticles were washed and centrifuged with deionized water for several times, then freeze-dried and stored at 4° C. for later use.

### Detection of Photothermal Conversion of PLGA-NPs

DdH2O, blank nanoparticles, 0.012 mg/mL free ICG, nanoparticles containing ICG and Fe₃O₄ (concentration of ICG is 0.012 mg/mL) and nanoparticles containing ICG and oleic acid modified Fe₃O₄ were continuously irradiated at 1.5 W/cm2 by 808 nm laser emitter. The results showed that PLGA nanoparticles containing ICG and oleic acid modified Fe₃O₄ had the strongest ability to maintain high temperature. Then, PLGA nanoparticles coated with oleic acid modified Fe₃O₄ and free oleic acid modified Fe₃O₄ were irradiated continuously. It was found that the two types of PLGA nanoparticles coated with Fe₃O₄ and free Fe₃O₄ had certain thermogenesis. But the heat production capacity is low. Then, we used intermittent irradiation method, every 3 minutes to raise the temperature to the highest point, and stopped the irradiation for 7 minutes to bring the temperature back to room temperature until the temperature no longer increased.

Preparation and Phenotype Analyses of NJ001-ICG/OA-Fe₃O₄@NP

The nanoparticles previously prepared have the ability to connect antibodies on the surface. After 2 mg ICG/OA-Fe₃O₄@PLGA was activated by EDC/NHS method, 5 μg hrP-conjugated antibody was linked by amide reaction. After the reaction was completed, the amount of hrP-conjugated antibody on the surface of the microspheres was calculated by detecting the chromogenic degree of the nanoparticles to the substrate.

### Ex Vivo PTT and Cell Viability

1) Cytotoxicity test: Spc-a1 cells were inoculated into 96-well plates. They were co-incubated with NJ001-ICG/OA-Fe₃O₄@NP, NJ001-NP, ICG/OA-Fe₃O₄@NP, ICG/OA-Fe₃O₄, blank-NP and PBS at a certain concentration gradient (1 mg/mL, 2 mg/mL, 4 mg/mL or 8 mg/mL), respectively. After 24 h culture, the cells were washed with PBS for three times and irradiated with 808 nm, 1 W/cm2 near infrared light for 5 min. The cell survival rate was detected by MTT method. 100 μL medium containing 10% MTT (5 mg/mL) was added to each well and cultured at 37° C. in dark for 4 h. Then, the medium was removed and 150 μL dimethyl sulfoxide was added to each cavity. The medium was shocked slowly at dark room temperature for 10 min. 2) Apoptosis experiment: Spc-a1 cells were inoculated into 96-well plates. They were co-incubated with NJ001-ICG/OA-Fe₃O₄@NP, NJ001-NP, ICG/OA-Fe₃O₄@NP, ICG/OA-Fe₃O₄, blank-NP and PBS at a certain concentration gradient (1 mg/mL, 2 mg/mL, 4 mg/mL or 8 mg/mL), respectively. After 24 h culture, PBS was washed three times and irradiated with 808 nm, 1 W/cm2 near infrared light for 5 min. The treated cells were collected and stained with apoptosis kit. The fluorescence signal was recorded by flow cytometry, and the signal intensity was quantitatively analyzed by Flowjo software.

### Tumor-Bearing Mouse Model and Tumor Inhibition

Thirty-four Four-week-old male BALB/c nude mice were maintained in a pathogen-free environment. To establish an orthotopic xlung tumor implantation mouse model, SPC-A1-luc cells were harvested in the logarithmic growth phase, counted, and resuspended in phosphate-buffed saline (PBS) to a final density of 5×10⁷ cells/mL. Then, 5×10⁶ SPC-A1-LUC cells were suspended in 0.1 mL sterile PBS, and 5×10⁶ SPC-A1-LUC cells suspended in 0.1 mL sterile PBS were injected into mice intravenously via tail vein. All animal experiments were approved by the Animal Protection and Use Committee of Nanjing Medical University.

### In Vivo Imaging

In Tumor-bearing mouse models, the bioluminescent imaging system was used to detect lung bioluminescence signal values every week. Each nude mouse was intraperitoneally injected with luciferin substrate at a dose of 150 mg/kg. In vivo imaging was performed 10-15 min after isoflurane anesthesia, and the IVIS software of the system was used 2000 System (purchased from Caliper) to detect the bioluminescence signal intensity of lung tumors. After in vivo imaging, the tumor and liver, spleen, kidney, heart, lung and other major organs were dissected for in vitro fluorescence detection.

### Micro-Computed Tomography (Micro-CT)

Orthotopic mouse models were imaged with in vivo imaging of small animals to monitor tumor growth one week after SPC-A1-luc cell inoculation. Nine mice with no significant difference in tumor size were selected for the following micro-CT scans. Three mice were intravenously injected with NJ001-ICG/OA-Fe₃O₄@NP, three mice were injected with ICG/OA-Fe₃O₄@NP, two mice were injected with NJ001-NP, and one mouse was injected with Blank-NP. Micro-CT scans was acquired 2 h after injection to determine the tumor lesion and size. Micro-CT was performed on a SkyScan 1176 (SkyScan NV, Kontich, Belgium), a small animal imager, at 50 kV and 490 μA. A total of 360° views were acquired at 1° angle increments, each for an exposure time of 120 ms, to give a resolution of 35 μm using external respiratory gating. SkyScan soft-ware was used for multiplanar and 3D image reconstruction.

### Example 2. Synthesis and Characterization of ICG/OA-Fe₃O₄@NPs

To synthesize an effectiveness photothermal agents, we explore the synergistic effect of ICG and OA-Fe₃O₄. Various photothermal agents were exposed to 808 nm NIR laser irradiation with a power density of 1.0 W/cm² for 10 min. Aqueous suspensions of ICG/OA-Fe₃O₄@NPs, mixture of free ICG and OA-Fe₃O₄ (1 mL solution with 12 μg/mL ICG or 11.6 μg/mL Fe) reach the maximum temperatures of 45.9° C., 46.6° C. In contrast, under the same concentration and laser irradiation conditions, the maximum temperatures achieved in ICG@NP, free ICG and OA-Fe₃O₄@NP were 43.5° C., 41.8° C. and 31.9° C., while no obvious temperature change was observed in blank-NP and deionized water.

The higher maximum temperature observed for the ICG/OA-Fe₃O₄@NPs and the mixture of ICG and OA-Fe₃O₄ NPs indicates that the combination of ICG and OA-Fe₃O₄ NPs may be more effective than pure ICG or Fe₃O₄ NPs in PTT.

We then compared the photothermal stability of various photothermal agents to check the enhanced stability of photothermal agents encapsulated in polymer by nanotechnology. ICG@NPs, free ICG, ICG/OA-Fe₃O₄@NPs, free ICG/OA-Fe₃O₄ were irradiated with the NIR laser for 3 min (Laser ON), followed by natural cooling for 7 min (Laser OFF). After each of the six laser ON/OFF cycles, the maxim temperature of free ICG were 40.9° C., 37.1° C., 33.8° C., 32.8° C., 30.7° C., 30.1° C., while ICG@NP were 40.0° C., 38.2° C., 38.6° C., 36.5° C., 35.0° C., 33.9° C. The maxim temperature of mixture of free ICG and OA-Fe₃O₄ were 43.6° C., 42.1° C., 40.8° C., 40.5° C., 38.9° C., 35.8° C., while ICG/OA-Fe₃O₄@NPs were 45.6° C., 45.5° C., 44.8° C., 45.1° C., 45.1° C., 45.3° C.

The stable temperature of the ICG@NP and ICG/OA-Fe₃O₄@NPs indicated that ICG loaded in the polymeric layers NPs show greater photostability than the free ICG molecules. The temperature increases in the ICG/OA-Fe₃O₄@NPs solution were greater than those in the ICG@NP solution, which showed that OA-Fe₃O₄ in the former NPs also contributed to the photothermal conversion efficiency and photothermal stability.

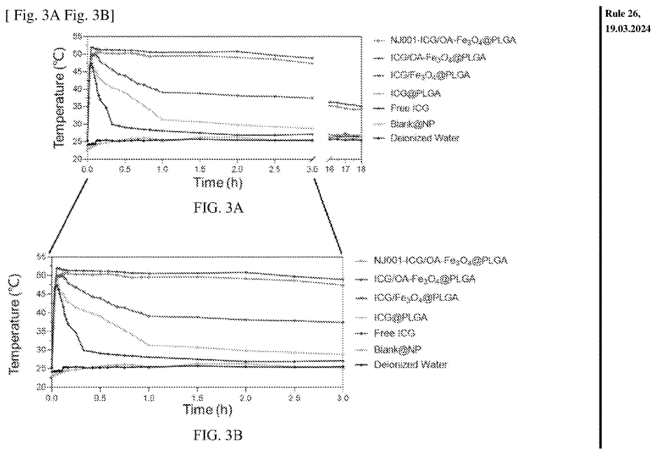

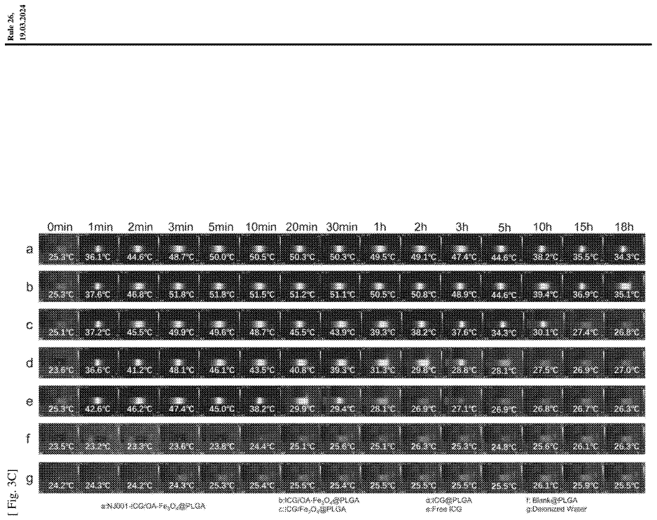

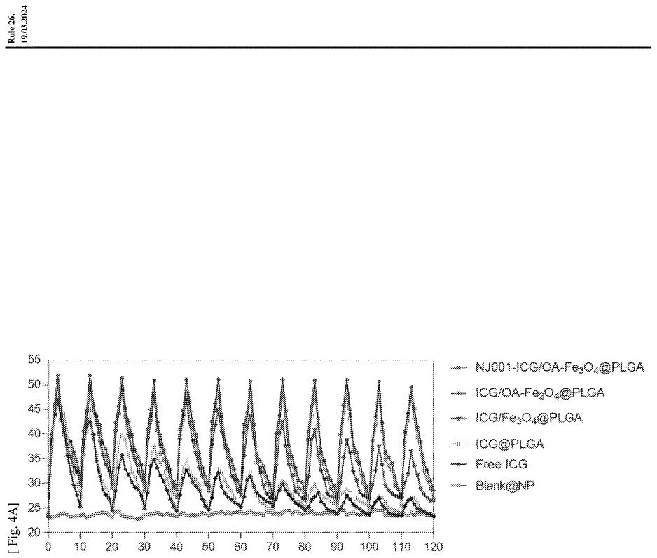

To evaluate its photothermal conversion performance, ICG/OA-Fe₃O₄@PLGA solutions were irradiated with an 808 nm NIR laser (1.5 W/cm², FIGS. 3A-3C). The results showed that the temperature of ICG/OA-Fe₃O₄@PLGA (500 μg mL-1) solution increased to 51.9° C. within 3 min and maintained above 45° C. for 2.5 h, while ICG/Fe₃O₄@PLGA, ICG@PLGA and free ICG also reached to 49.9° C., 48.1° C. and 47.3 respectively, but high temperature was maintained for only a few minutes. The maximal advantage of ICG/OA-Fe₃O₄@PLGA in photothermal conversion was that high temperature can be maintained for prolonged periods of time. No significant temperature changes were found in Blank-NP solution and DI water. Only negligible temperature increase of Blank-NP solution and DI water were found.

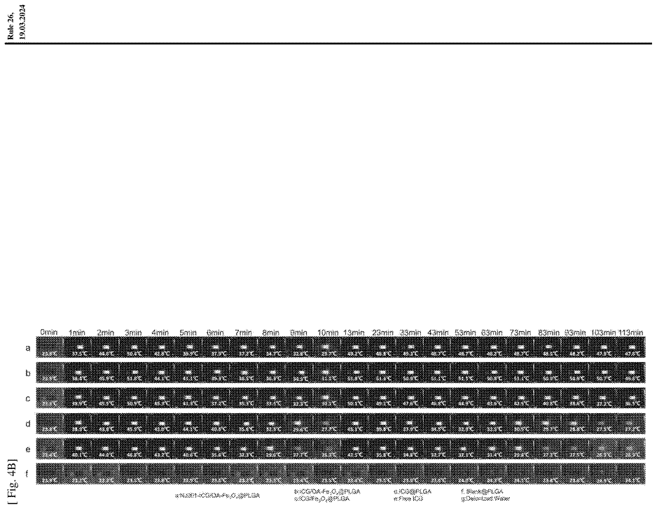

Furthermore, to investigate the photothermal conversion stability of ICG/OA-Fe₃O₄@PLGA for practical applications, solutions of ICG/OA-Fe₃O₄@PLGA, ICG/Fe₃O₄@PLGA, ICG@PLGA, Blank-NPs and free ICG were irradiated with the NIR laser for 3 min (Laser ON), followed by natural cooling for 7 min (Laser OFF). We found no significant decay in the heating ability of ICG/OA-Fe₃O₄@PLGA NPs during 12 cycles (FIGS. 4A-4B), which indicates that it could generate sufficient heat efficiently and consistently under NIR irradiation without signs of fatigue.

### Example 3. Characterization of Targeted ICG/OA-Fe₃O₄@NPs

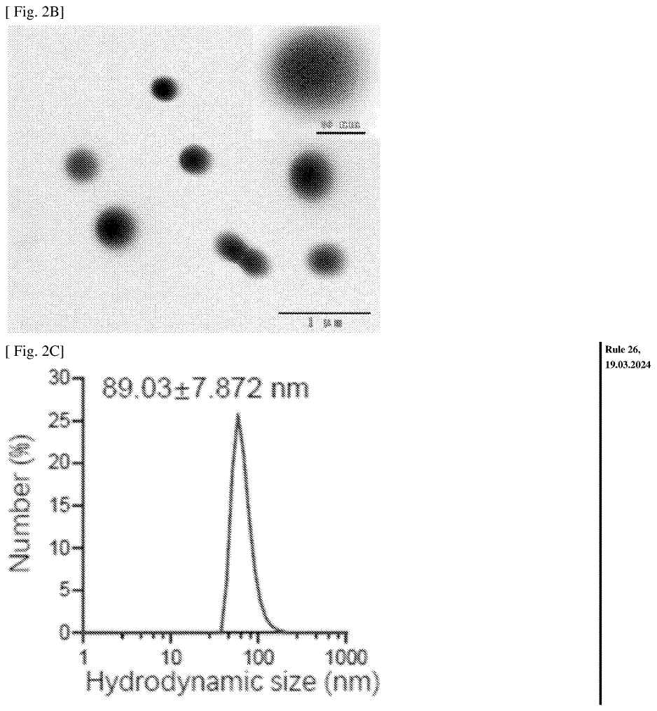

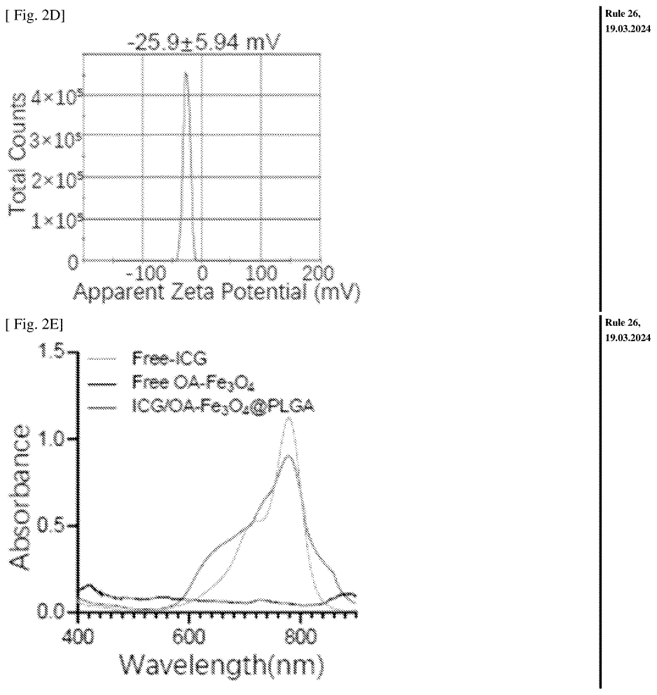

The characterization of the nanoparticles are shown in FIGS. 2A-2E. Particle size detection shows that the average particle size of the prepared nanoparticles is 70-90 nm. After ICG/Fe₃O₄@PLGA was placed for 7 days and 30 days, particle size was detected, and no significant change in particle size was observed. The Zeta potential on the surface of the nanoparticles is-22.5 mV, indicating that the nanoparticles have good dispersion, rich surface locking groups, and can load a large amount of protein. General scanning electron microscopy showed that the ICG/Fe₃O₄@PLGA prepared was regular round, smooth surface and uniform particle size. Transmission electron microscopy (TEM) showed that black particles were evenly distributed in the nanoparticle and Fe₃O₄ was successfully encapsulated in the nanoparticle. Uv-vis spectra showed that the maximum absorption peak of free ICG and ICG/Fe₃O₄@PLGA ranged from 790-810 nm. The maximum absorption wavelength of free ICG was consistent with previous reports, and the absorption wavelength of ICG/Fe₃O₄@PLGA was corrected by Blank-NP.

As shown in FIGS. 3A-3C, ICG/OA-Fe₃O₄@PLGA has constant high photothermal conversion efficiency.

FIGS. 4A-4B shows the photothermal stability of ICG/OA-Fe₃O₄@PLGA. The temperature of the photothermal nanoparticles remained stable for at least 8 hours.

### Example 4. In Vivo PTT for Tumor Ablation

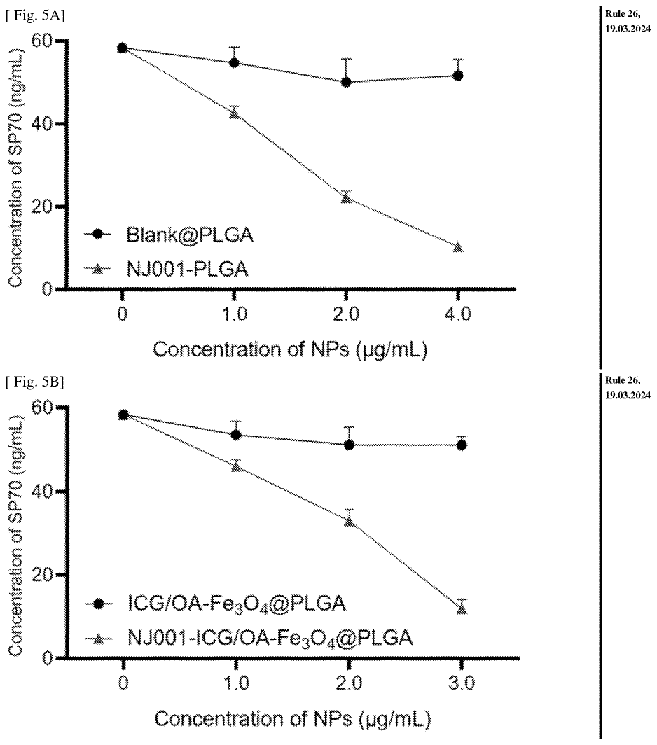

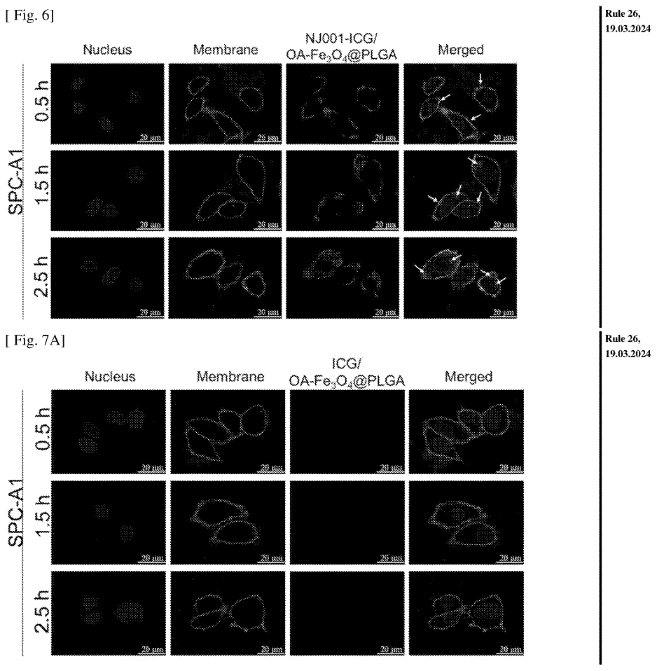

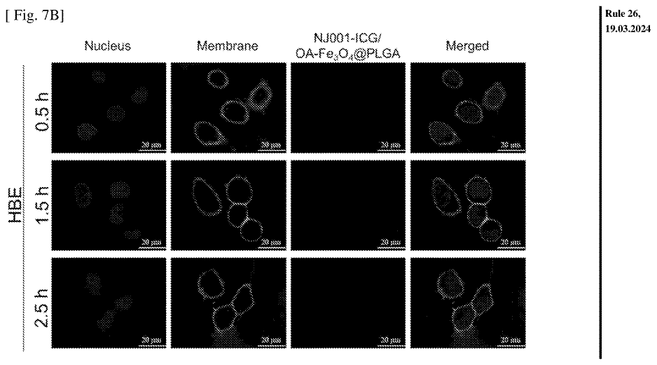

FIGS. 5-7B show the accumulation and distribution of the nanoparticles that specifically target a lung tumor antigen SP70. The tumor-targeting ability of the nanoparticle were obtained through covalently cross-linking novel tumor marker SP70 specific antibodies NJ001 to ICG/OA-Fe₃O₄@PLGA using EDC/NHS method. NJ001 labeled ICG/OA-Fe₃O₄@PLGA (NJ001-ICG/OA-Fe₃O₄@PLGA) cocultured with SPC-A1 cell culture supernatant which is rich in SP70 protein. After centrifuge, the residual SP70 in supernatant were measured. The residual SP70 in supernatant generally decrease (albeit slowly) in response to NJ001 concentration changes, while non-targeted ICG/OA-Fe₃O₄@PLGA without NJ001 cannot bind SP70 (FIG. 5). To detect the binding ability of targeted nanoparticles to SP70 positive cells, the different lung cell lines with or without SP70 expression was cocultured with NJ001-ICG/OA-Fe₃O₄@PLGA or ICG/OA-Fe₃O₄@PLGA. After cocultured for 30 min, the cells were washed and continued to be cultured in their respective culture conditions. Intracellular distributions of NJ001-ICG/OA-Fe₃O₄@PLGA in SPC-A1 cells detected by confocal microscopy. As shown in FIGS. 7A-7B, the SPC-A1 cells (high expression of SP70) had no binding and uptake of ICG/OA-Fe₃O₄@PLGA and the HBE cells (no SP70 expression) had no binding and uptake of NJ001-ICG/OA-Fe₃O₄@PLGA. In contrast, NJ001-ICG/OA-Fe₃O₄@PLGA binding to SPC-A1 cells (high expression of SP70) after cocultured for 30 min, and as the time-lapse culturing continued, these NPs gradually uptake by SPC-A1 (FIG. 6).

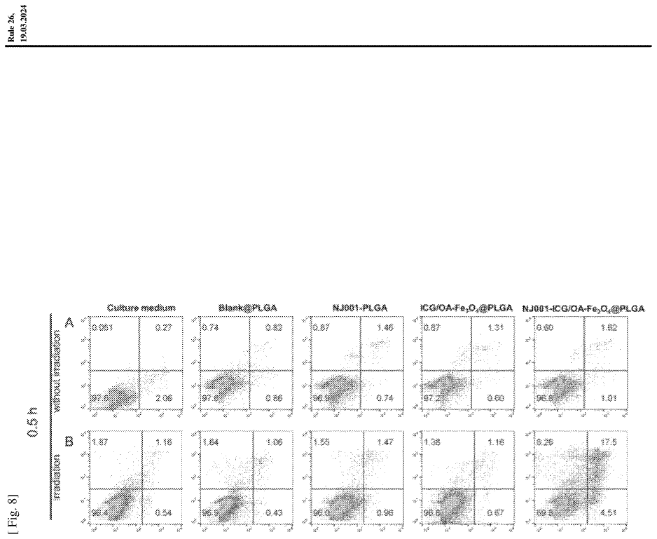

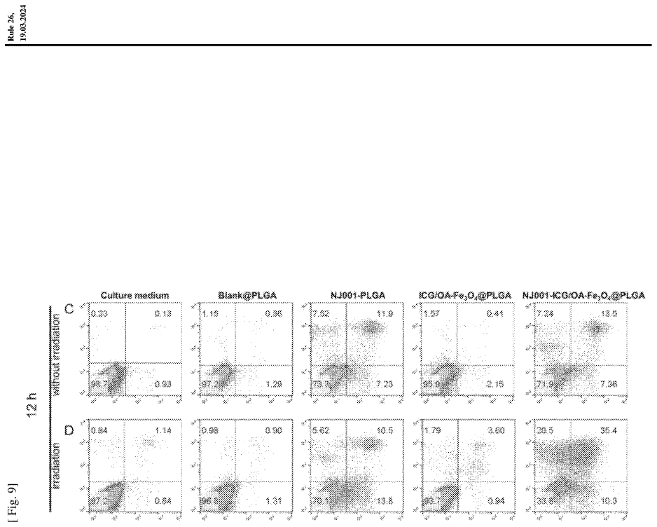

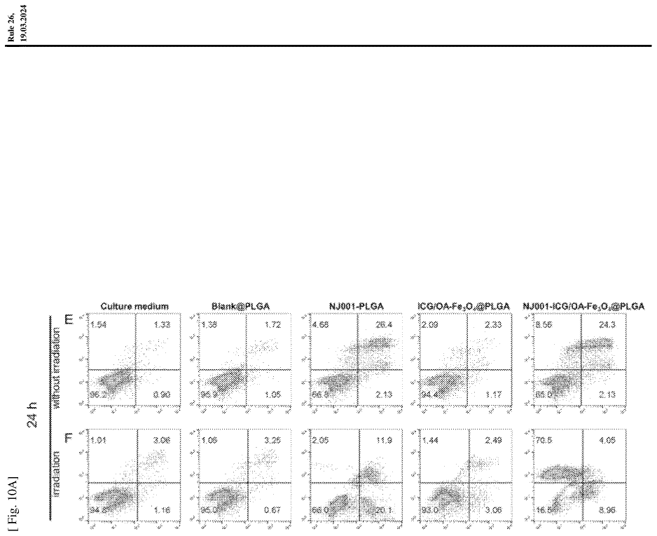

FIGS. 8-10B show the in vitro tumor cell ablation by the nanoparticles. In order to compare the strength of the apoptotic effect in NJ001-ICG/OA-Fe₃O₄@PLGA, NJ001-NP and ICG/OA-Fe₃O₄@PLGA, Blank-NP, flow cytometry analysis of Annexin V-FITC and PI was performed. To examine NJ001 and ICG induced apoptosis in SPC-A1 cells quantitatively, the cells were exposed to various NPs with or without NJ001 and ICG for 30 min, and the percentage of cell apoptosis was determined by flow cytometry analysis at 0, 12, 24 hours after NIR irradiation. Cells were incubated for the same time but without irradiation as control. In the non-irradiated groups, the apoptosis rate of cells treated with NJ001-labeled particles was higher than that of cells treated with NJ001-unlabeled particles, and increased with the extension of culture time. The percentage of apoptosis of NJ001-ICG/OA-Fe₃O₄@PLGA treated cells was 35.0±3.5% within 24 hrs without irradiation. This difference was significantly improved when the tumor cells were killed by synergistic antibody and photothermal action. The percentage of apoptosis of NJ001-ICG/OA-Fe₃O₄@PLGA treated cells was 83.5±2.8% within 24 hrs with NIR irradiation. The results indicate that NJ001 labeled ICG/OA-Fe₃O₄@PLGA has higher cytotoxicity rate compared to those of ICG/OA-Fe₃O₄@PLGA (non-targeted) and NJ001-NP (non-thermal) NPs (FIGS. 8-10B).

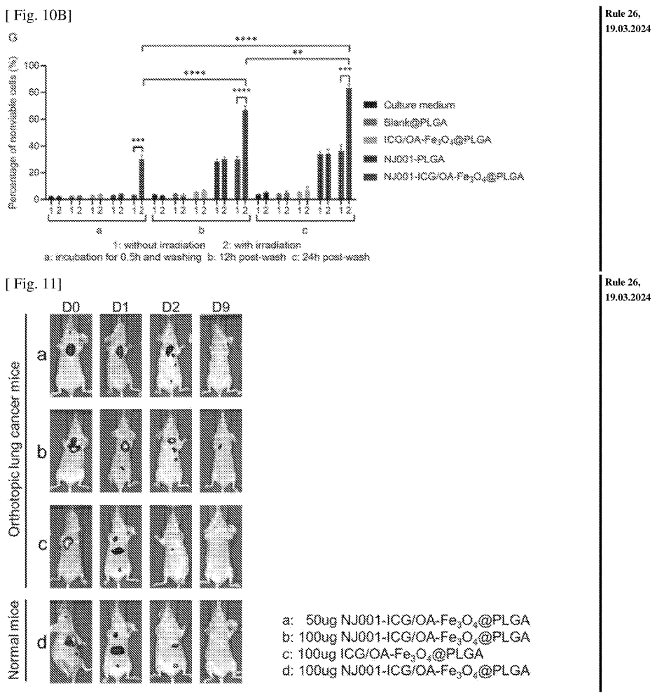

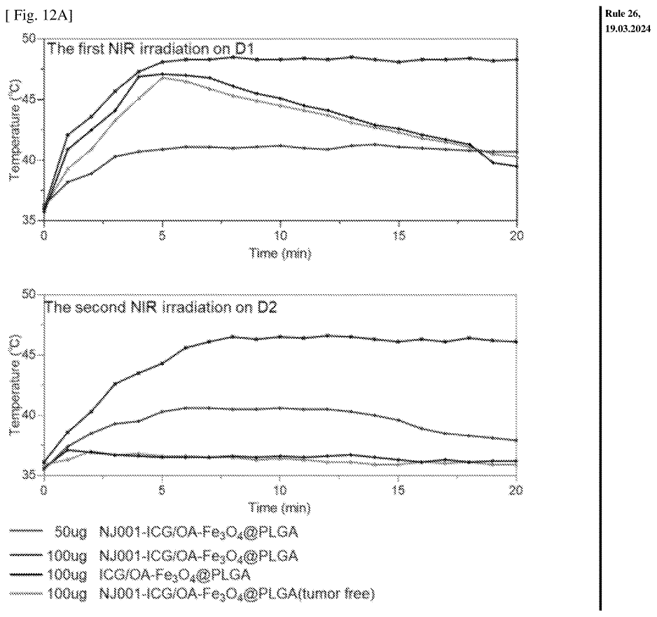

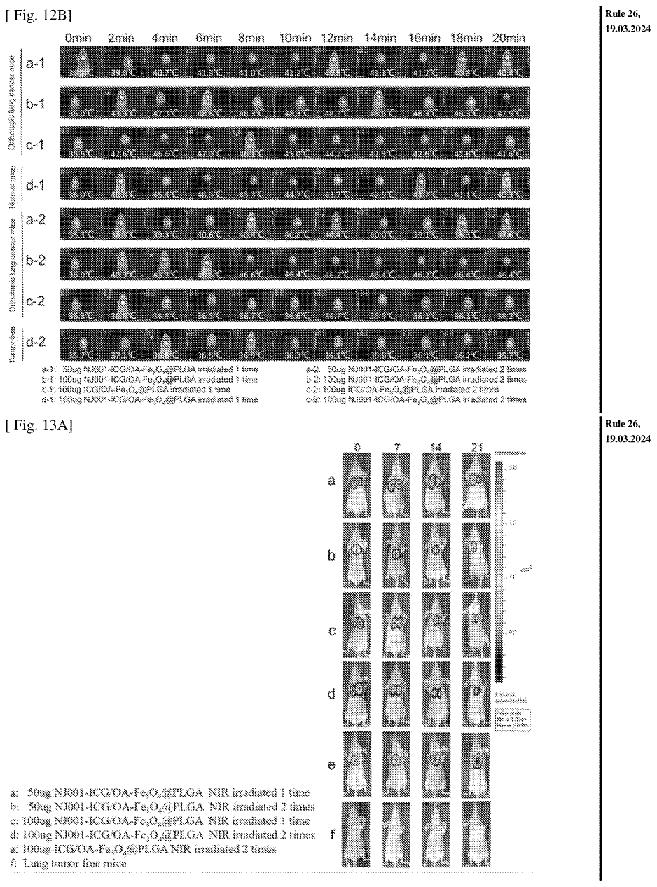

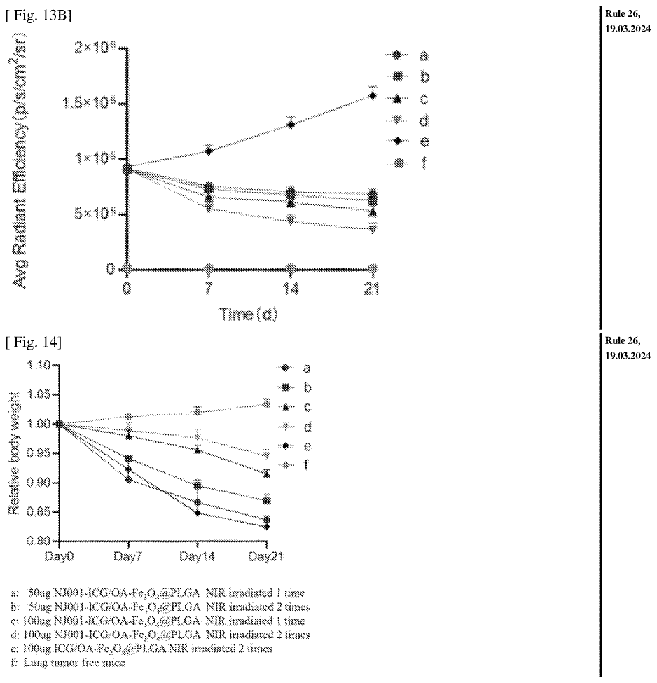

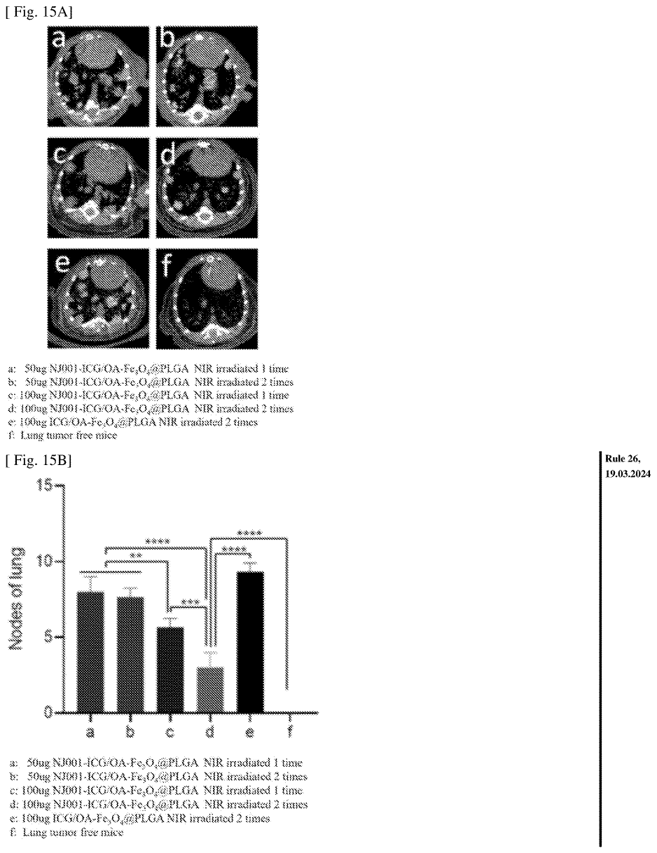

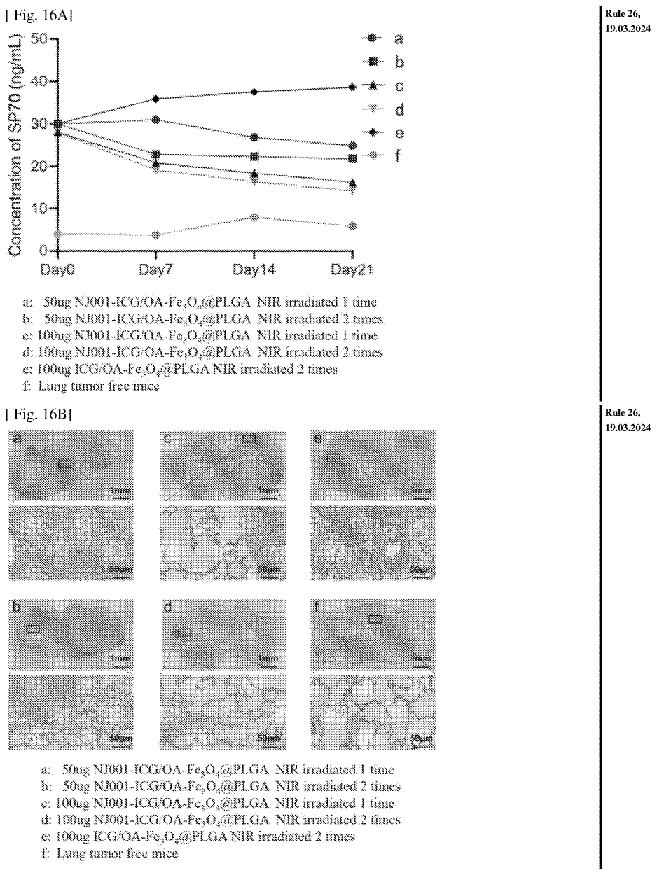

FIGS. 11-18 show the in vivo tumor cell ablation by the nanoparticles. Specifically, the distribution of NPs in the lung were investigated. Following inhalation of the various types of NPs, the mice that inhaled different formulations were monitored at 24, 48, and 72 hours post inhalation using an in vivo imaging system (IVIS). Excitingly, with the assistance of NJ001, targeted NPs exhibited greatly improved retention ability. It was discovered that the NJ001-ICG/OA-Fe₃O₄@PLGA showed the best retention effect, as the fluorescence signals of ICG from NPs in the mice treated with NJ001-ICG/OA-Fe₃O₄@PLGA remained the strongest at all time points demonstrating that the SP70 targeted antibody NJ001 played an indispensable role in NPs accumulation and protecting NPs from rapid clearance. In contrast, ICG/OA-Fe₃O₄@PLGA particles are quickly cleared from the lung.

Thus, such an NJ001-ICG/OA-Fe₃O₄@PLGA nanocomposite can be the ideal photothermal agents to conduct photothermal ablation of the tumor after inhalation (FIG. 11).

### Example 5. The Nanoparticles have Minimum Toxicity to Normal Tissues In Vivo

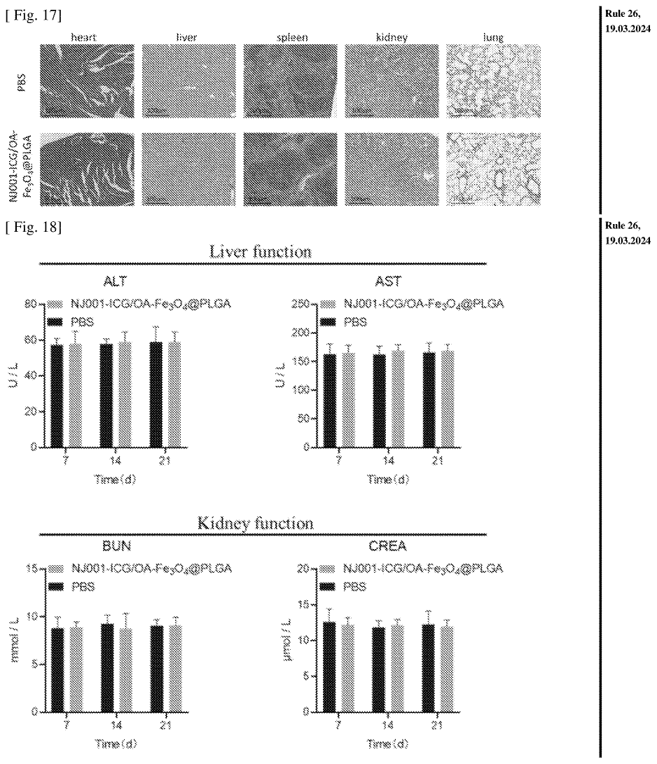

On days 14, 21, and 28, the functions of liver and kidney of nude mice were not impaired significantly by NJ001-ICG/OA-Fe₃O₄@PLGA treatment as monitored by routine serum biochemical tests of peripheral blood. No visible damage was observed in the tissue sections of spleen, kidney, liver, heart, and lungs after NJ001-ICG/OA-Fe₃O₄@PLGA treatment when compared with the PBS treatment, as analyzed by H&E staining at the same time points (FIGS. 17 and 18). Both functional and histopathological analyses suggest that the NJ001-ICG/OA-Fe₃O₄@PLGA treatment did not cause apparent organ toxicity at the longtime points in the orthotopic lung tumor mice.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising:
a photothermal agent, and one or more core particles,
wherein the photothermal agent and the core particles are encapsulated in a nanoparticle made from a biodegradable material.

2. The composition of claim 1, wherein the core particles are solid particles.

3. The composition of claim 2, wherein the metal particles are Fe₃O₄ particles.

4. The composition of any one of the preceding claims, wherein the core particles are surface-modified for the attachment of the photothermal agent.

5. The composition of claim 4, wherein the core particles are modified on the surface by oleic acid molecules.

6. The composition of claim 5, wherein the photothermal agent is attached to the surface of the core particles through hydrophobic interactions with the oleic acid molecules.

7. The composition of any one of the preceding claims, wherein the core particles have a diameter of about 10 nm to about 40 nm.

8. The composition of any one of the preceding claims, wherein the composition comprises about 1 to about 10 core particles.

9. The composition of any one of the preceding claims, wherein the photothermal agent is selected from the group consisting indocyanine green (ICG), polydopamine, polyaniline (PANI), and polypyrrole (PPY).

10. The composition of claim 9, wherein the photothermal agent is indocyanine green (ICG).

11. The composition of any one of the preceding claims, further comprising a therapeutic agent, wherein the therapeutic agent is encapsulated in the nanoparticle.

12. The composition of any of the preceding claims, wherein the therapeutic agent is an anti-cancer therapeutic agent.

13. The composition of any one of the preceding claims, wherein the composition further comprises a tumor-targeting molecule attached to the surface of the nanoparticle.

14. The composition of claim 13, wherein the tumor-targeting molecule is an antibody, or antigen-binding fragment thereof, that binds to a tumor-specific antigen.

15. The composition of any one of the preceding claims, wherein the diameter of the nanoparticle is about 70 nm to about 120 nm.

16. A method of producing the composition of any of the preceding claims, wherein the method comprises:
(a) contacting one or more core particles with the photothermal agent, thereby attaching the photothermal agent to the surface of the core particles;
(b) preparing a double emulsion comprising the biodegradable material; and
(c) contacting the product of step (a) with the double emulsion prepared in step (b), thereby producing the nanoparticles encapsulating the core particles;
thereby producing the composition.

17. The method of claim 16, wherein the biodegradable material is poly (lactic-co-glycolic acid) (PLGA).

18. The method of claim 16 or 17, wherein the double emulsion further comprises polyvinyl alcohol (PVA).

19. The method of any one of claims 16-18, wherein the method further comprises washing and/or freezing the nanoparticles.

20. A method of imaging a tumor tissue in a subject in vivo, the method comprising:
(a) administering an amount of the composition of any one of claims 1-15 to the subject;
(b) exciting the photothermal agent of the composition; and
(c) detecting the emitted fluorescent signals,
thereby imaging the tumor tissue.

21. The method of claim 20, wherein the quality of the imaging is enhanced compared to an imaging without the administration of the composition.

22. A method of diagnosing a tumor in a subject, the method comprising:
(a) administering an amount of the composition of any one of claims 1-15 to the subject;
(b) exciting the photothermal agent of the composition; and
(c) detecting the presence or absence of emitted fluorescent signals, thereby diagnosing the tumor.

23. The method of claim 22, further comprising recording the intensity of the emitted fluorescent signals.

24. A method of treating a subject having tumor, the method comprising:
(a) administering an amount of the composition of any one of claims 1-15 to the subject;
(b) exciting the photothermal agent of the composition, thereby increasing the temperature of the tumor cells that has internalized the composition and damaging the tumor cells,
thereby treating the subject having the tumor.

25. The method of claim 24, further comprising treating the tumor with the therapeutic agent of the composition.

26. The method of claim 25, wherein the therapeutic agent is a chemotherapy agent selected from the group consisting of paclitaxel, chlorambucil, cyclophosphamide, thiotepa, busulfan, purine antagonists, pyrimidine antagonists, folate antagonists, actinomycin D, doxorubicin, mitomycin, doxorubicin, mitoxantrone, and bleomycin.

27. The method of any one of claims 20-26, wherein the tumor is a solid tumor.

28. The method of any one of claims 20-27, wherein the photothermal agent is excited using a near-infrared (NIR) light.

29. The method of any one of claims 20-28, further comprising targeting a specific tumor type by the tumor-targeting molecule of the composition.

30. The method of any one of claims 20-29, wherein the tumor is lung cancer.

31. The method of any one of claims 20-30, wherein the subject is a human subject.

32. The method of any one of claims 20-31, wherein the administration is selected from the group consisting of intravenous injection, intraperitoneal injection, oral administration, oral transmucosal delivery, subcutaneous administration, intradermal administration, and transdermal administration.

33. The method of any one of claims 24-32, wherein the photothermal agent is degraded after emitting heat.

34. The method of claim 33, wherein the heat emitted from the photothermal agent is transmitted to the core particles, thereby increasing the heating duration.

35. The method of any one of claims 24-34, wherein the heating of the tumor cells induces the apoptosis of the tumor cells.

36. The method of any one of claims 20-35, wherein the composition is internalized into the tumor cells through non-specific endocytosis.

37. The method of any one of claims 20-35, wherein the composition is internalized into the tumor cells through specific recognition of tumor antigens.

38. The method of any one of claims 24-35, wherein the heating of the tumor cells is repeated at least once.

39. The method of claim 38, wherein the tumor cells are heated to about 40° C. to about 45° C.

40. A method of evaluating a therapeutic effect in a subject undergoing treatment with one or more cancer therapy, the method comprising:
(a) administering an amount of the composition of any one of claims 1-15 to the subject;
(b) imaging a tumor area and determining the tumor size;
(c) repeating the imaging and the determining of the tumor size at a later time point; and
(d) evaluating the therapeutic effect based on the tumor size from the different time points;
thereby evaluating the therapeutic effect in the subject.

41. The method of claim 40, further comprising imaging and determining the tumor size before the subject is treated with the cancer therapy.

42. The method of claim 40 or 41, wherein the repeating of the imaging and determining of the tumor size is performed after about 1 week to about 1 year.

43. The method of any one of claims 40-42, wherein the imaging is X-ray computed tomography (CT), magnetic resonance imaging (MRI), magnetic particle imaging (MPI), photoacoustic imaging (PA), ultrasound imaging (US), fluorescent imaging (FL), or a combination thereof.
